(19) United States
(12) Patent Application Publication
(10) Pub. No.: US 2016/0178618 A1
(43) Pub. Date: Jun. 23, 2016

(54) 3D TISSUE MODEL FOR SPATIALLY CORRELATED ANALYSIS OF BIOCHEMICAL, PHYSIOLOGICAL AND METABOLIC MICRO-ENVIRONMENTS

(71) Applicant: STC.UNM, Albuquerque, NM (US)

(72) Inventors: James P. Freyer, Placitas, NM (US); Andrew P. Shreve, Santa Fe, NM (US); Jacqueline DeLora, Albuquerque, NM (US)

(21) Appl. No.: 14/973,303

(22) Filed: Dec. 17, 2015

Related U.S. Application Data
(60) Provisional application No. 62/093,214, filed on Dec. 17, 2014.

(51) Int. Cl. G01N 33/50; G01N 33/574; C12M 1/00; C12M 1/34; C12M 3/00

(52) U.S. Cl. CPC G01N 33/5082; C12M 41/46; C12M 41/38; C12M 41/26; C12M 21/08; C12M 29/10; G01N 33/574

(57) ABSTRACT

Disclosed is a perfusion device and methods of use including a generally cylindrical body and a packed bed of nanosensor-cell embedded matrix spheres (nanoCEMS) disposed between layers of inert microspheres. A concentration of a molecule of interest can be established within the perfusion device to effect the cellular and chemical microenvironment of the nanoCEMS, the nanoCEMS in turn creating their own concentration gradients nutrients and waste products in response, which can be measured by nanosensors. The collected measurements can be applied to a transport model to calculate concentrations of various molecules at discreet locations in the perfusion chamber. Also disclosed is a method for making nanoCEMS by mixing a polymeric mixture with a crosslinking solution and dispersed through a nested dispensing device such that mixing of the two mixtures occurs in air. A piezoelectric transducer coupled to the dispensing device controls the droplet formation rate.

### CROSS REFERENCE TO RELATED PATENTS

This application claims the benefit of U.S. Provisional Patent Application No. 62/093,214 filed Dec. 17, 2014, which is incorporated by reference.

### STATEMENT REGARDING FEDERALLY-SPONSORED RESEARCH OR DEVELOPMENT

This invention was made with government support under R01CA132629 and F31CA189682 awarded by NIH. The government has certain rights in the invention.

### FIELD OF THE INVENTION

The present disclosure relates generally to three dimensional tissue models. More specifically, the disclosure provides for a device and methods of use of cell-embedded matrix spheres containing nanosensors therein (nanoCEMS), as well as a means for producing the nanoCEMS, to allow measurements of spatial distributions of the chemical and cellular microenvironments.

### BACKGROUND OF THE INVENTION

The past 10 years has seen an explosive development of the field of three-dimensional cell culture. Most of this work has been directed at developing systems for investigating fundamental cell biology and cell-cell interactions in a more tissue-like environment or improving techniques for mass culture of mammalian cells. Surprisingly, relatively little work has been done to investigate the influence of the tissue environment on functional regulation of the cellular genome, in spite of the importance of this regulation in such diverse fields as tumor biology, artificial organ development and bioprocess control of bioreactors. A major explanation for this is the current lack of 3-D tissue model systems that are amenable to correlated measurement of microenvironmental, physiological, metabolic and functional genomics parameters.

One well studied 3-D model is the multicellular spheroid, a spherically-symmetric aggregate of cells which has been used extensively for studies of the effects of the local microenvironment on response to cancer therapy. This model has also proven very useful for studies of the relationship between the microenvironment and basic cellular physiology, such as proliferation, viability, energy metabolism and gene/protein expression. One of the major advantages of this experimental system is its spherical symmetry, which has allowed the development of methods for isolating cells from known locations within the 3-D structure for assay of microenvironmental effects on cellular physiology. However, due to their small size, spheroids are unsuitable for direct measurement of the chemical microenvironment except using specialized microelectrodes or destructive histochemistry.

Another model that is widely used for production purposes is the hollow-fiber bioreactor. A major disadvantage of this system is the inability to recover cells from known locations within the 3-D structure, precluding correlations between microenvironmental, physiological, metabolic and functional genomics measurements. Another recent development is the multicellular membrane, in which cells are grown in layers on top of a porous membrane. Although these have been useful for studying drug transport questions, no one has yet described methods either for measuring microenvironmental conditions within these structures nor for recovering cells from different locations. Numerous systems have been developed and commercialized for 3-D cell culture in a variety of natural and artificial matrix materials, both for basic research and for a variety of applications such as artificial organs, biosensors, drug screening and stem cell production.

The field of tumor biology has stimulated the majority of work on understanding the complex interactions between cells and their surrounding environment, particularly in the case of microenvironmental stress. Tumors develop large micro-regional variations in nutrients, growth factors and waste products due to an insufficient and chaotic blood supply, and this has been shown to alter tumor cell physiology such that the response to anticancer therapy is severely compromised. Numerous critical physiological and metabolic alterations are induced by the tumor microenvironment, including proliferation arrest, increased invasion, resistance to apoptosis, reduced mitochondrial function, decreased metabolic rates and altered protein synthesis . Due to the complexity of the tumor microenvironment, there is little mechanistic understanding of the regulation of any of these processes. A good illustrative example is tumor cell proliferation, which is commonly thought to be regulated by oxygen availability. Critical review of the work in this area shows that there is, at most, only a correlation between hypoxia and cell cycle arrest. This dogma is largely due to the development of sophisticated techniques for measuring hypoxia in tumors, and persists despite data clearly showing that factors other than oxygen actually regulate proliferation in tumor models and tumors. A clear understanding of the microenvironmental regulation of tumor cell physiology and metabolism will only come with improved systems for regulating and measuring the actual biochemical conditions surrounding cells.

Much work on microenvironmental regulation of gene and protein expression in mammalian cells has also been focused on tumor biology. It has been demonstrated that alterations in gene and protein expression induced by the local tumor microenvironment are involved in the generation of new blood vessels (angiogenesis), the resistance of cells to chemotherapeutic agents, the ability of tumors to invade normal tissues and move to other sites (metastasis), and the response to all forms of cancer therapy including surgery . Most of the evidence for these functional genomics alterations comes from immunohistochemical and in situ hybridization studies of tumor sections, a destructive, single time point analysis which points up a major limitation to our understanding of any complex tissue system. Although micro-regional alterations in gene and protein expression are known to occur in tumors, there is essentially no information concerning the regulation of the observed changes other than correlations with single variables like oxygen. In vitro work in which an individual variable is manipulated indicate that genomic and proteomic regulation is extremely complex, leading to often conflicting results in different systems. Much of this confusion results from the inability of current systems to measure, let alone control, multiple microenvironmental variables. It is clear that a major obstacle to advancing understanding of the regulation of the genome in multicellular systems is the lack of good experimental models.

A significant effort has been made to capture and predict the emerging biological behavior observed in experiments related to cancer progression and invasion through mathematically describing them with diffusion-reaction continuum equations. However, these models typically contain microenvironmental parameters that must be obtained from experimental data to enable more accurate descriptions and maximize the predictive power. Measuring or acquiring these parameters can be a difficult and arduous task, and much of the data available are scattered among different contributions over the last several decades. Particularly important in cancer research, parameters associated with concentration gradients of substrate molecules (e.g., oxygen and glucose etc.) are essential for determining a cell's phenotype. However, due to the difficulty of direct measurement, these parameters are often indirectly inferred from other more measureable cell distributions, but the data usually exhibits large variance and is not accurate because of other involved biological processes that lead to the observed cell distribution in response to the concentration gradient .

Accordingly, there is a need in the art for a 3-D tissue modeling system that will allow direct measurements of the spatial distributions of cellular and chemical microenvironments. The present invention addresses that need.

The present invention and its attributes and advantages will be further understood and appreciated with reference to the detailed description below of presently contemplated embodiments, taken in conjunction with the accompanying drawings.

### SUMMARY OF THE INVENTION

The present disclosure provides for a novel, three-dimensional (3D), in vitro tissue model that allows measurement of spatial distributions of microenvironmental parameters and directly correlates these with alterations in cellular physiology, metabolism and functional genomics. The system generally comprises a transparent, cylindrical perfusion chamber containing a packed bed of cell-embedded matrix spheres (CEMS) containing chemical nanosensors (nanoCEMS). The optically-responsive nanosensors are designed to report chemical conditions within the CEMS. An external optical analysis system measures light scatter and fluorescence signals from a small volume within the culture chamber. Perfusion of the device from one end produces chemical and cellular concentration gradients along the major axis due to metabolism and physiological adaptation. Translation of the optical analysis volume along the major axis provides real-time analysis of chemical and cellular gradients. Gradients in other cellular parameters (proliferation, function, gene/protein expression) are measured on cells and extracts obtained from nanoCEMS recovered from known locations within the chamber. Manipulation of initial conditions (e.g. perfusion rate, nutrient concentrations, cell types and concentrations) generates different biochemical, cellular and metabolic gradients. After starting perfusion with a packed bed of uniform nanoCEMS, the gradients within the device evolve as the cells adapt to their local microenvironment. Gradients in metabolism are measured by fitting the chemical and cellular measurements to a transport model to derive spatially-defined consumption/production rates. The design further comprises in-line chemical monitoring of the input and output perfusate, providing data on the bulk metabolism within the culture.

Accordingly, the present disclosure provides for a perfusion device comprising a chamber body having an input and an output, a removable screen adjacent the output, a plunger and screen, and a first layer of inert microspheres and a second layer of inert microsphere, and a layer of nanosensor-cell embedded matrix spheres (nanoCEMS) disposed there between, wherein the nanoCEMS comprise a crosslinked polymer matrix with entrapped cells and nanosensors.

In some embodiments, the polymer matrix comprises at least one polymer, biopolymer, ECM protein, or a combination thereof that is crosslinked via an ionic crosslinker. In further embodiments, the polymer is sodium alginate or calcium alginate, and the crosslinker is calcium ions.

In some embodiments, the entrapped cells comprise normal cells, stem cells, immortalized cells, cancer cells, genetically engineered cells, patient derived cells or a combination thereof. In other embodiments, a co-culture of two or more different cell types is encapsulated in the nanoCEMS, with each different cell type labeled with a different fluorescent marker (e.g. an inert membrane dye or a fluorescent protein). This allows separation of the co-culture into its constituent cell types, and provides a direct comparison of metabolism and cellular physiology of the two cell types in exactly the same microenvironments.

In further embodiments, the nanosensor is a fluorophore, nanoparticle, quantum dot or Cornell dots that detects oxygen concentration, carbon dioxide concentration, pH levels, metabolites, catabolites, secreted proteins, or ligand binding. The addition of a molecule to the input establishes a gradient of the molecule throughout the perfusion device. In some embodiments, the molecule is a metabolite such as glucose, lactate, glutamine, or a combination thereof.

Also provided is a method for measuring chemical and cell microenvironments comprising perfusing a fluid such as a media or perfusion fluid through a perfusion chamber as disclosed herein and adding a biological compound to the fluid to modify the chemical and cell microenvironments of the nanoCEMS by exposing the nanoCEMS to the biological compound.

In some embodiments, the nanoCEMS comprise normal cells, stem cells, immortalized cells, cancer cells, genetically engineered cells, patient derived cells or a combination thereof.

In some embodiments, the nanosensor is a fluorophore, nanoparticle, quantum dot, electrode or Cornell dots capable of detecting oxygen concentration, carbon dioxide concentration, pH levels, metabolites, catabolites, nutrients, waste products, secreted proteins, or ligand binding.

The nanosensor emits a signal that is detectable by direct optical interrogation such as epi-fluorescence, or via FRAP, Raman spectroscopy, Surface Enhanced Raman Spectroscopy or the like. In some embodiments, electrodes positioned at the input and output can measure the concentration of a compound such as a metabolite, pH or other parameter, and by comparing the measurements at the input and output, detect an overall change in the parameter. Other embodiments measure the concentration of one or more solutes secreted by the cells in the nanoCEMS in response to exposure to biological compound such as nutrients and waste products. Other solutes include, but are not limited to deoxyribonucleic acid, ribonucleic acid, proteins, carbohydrates, lipids, small peptides, or other organic or synthetic molecules, or any combination thereof. Still further embodiments measure the concentration gradient of the added biological compound or drug.

In some embodiments, the method further comprising the step of extruding the nanoCEMS and subjecting the extruded nanoCEMS to a biological assay wherein the biological assay can include Polymerase Chain Reaction, RT-PCR, ELISA assays, in situ hybridization, elastic light scattering, flow cytometry, microarrays or mass spectroscopy analysis, or a combination thereof.

In further embodiments, the nanosensor measurements as disclosed herein is fit into a cellular and chemical transport model. The transport model uses: 1) the measured concentrations of various solutes in the perfusion chamber at discreet locations; 2) the measured concentration of cells at discrete locations; and 3) the perfusate flow rate, to then calculate the metabolic activity as a function of location in the device (e.g. with oxygen as the measured metabolite, this method derives the oxygen consumption rate as a function of location within the chamber).

Also provided is a method for producing matrix spheres comprising the steps of providing a polymer mixture comprising at least one polymer and a ionic crosslinking mixture, pumping the mixtures into a pressurized capillary where the pressurized capillary has an inner chamber with an inner dispensing nozzle and an outer chamber with an outer dispensing nozzle, mixing in air the polymer mixture and the crosslinking mixture upon dispensing from the inner dispensing nozzle and the outer dispensing nozzle, where the crosslinking of the polymer mixture occurs in air to form the microspheres and the microspheres are captured in a receiving solution. The size of the microspheres is controlled by the vibration of a piezoelectric transducer coupled to the capillary device. In some instances, the piezoelectric transducer vibrates in a range of 2-10 kHz to produce microsphere droplets in a range of 50-150 micrometers.

In some embodiments, the polymer is alginate that is crosslinked with an ionic crosslinker such as calcium ions. The polymer mixture may further comprises a cell that is a normal cells, stem cells, immortalized cells, cancer cells, genetically engineered cells, patient derived cells or a combination thereof. The polymer mixture may further comprises a nanosensor, wherein the nanosensor is a fluorophore, nanoparticle, quantum dot or Cornell dots. The nanosensor may be directly conjugated to the polymer.

In still further embodiments, the crosslinking solution further comprises an interface stabilizing agent such as dextran or pluronic F-127 where the crosslinking solution forms an outer shell surrounding the polymer mixture upon crosslinking. In other embodiments, the polymer solution further comprises at least one other polymer such as poly ethylene glycol, or an ECM component.

### BRIEF DESCRIPTION OF THE DRAWINGS

The preferred embodiments of the invention will be described in conjunction with the appended drawings provided to illustrate and not to the limit the invention, where like designations denote like elements, and in which:

FIG. 1 illustrates a diagram of a perfusion device illustrating flow across nanoCEMS, resulting in chemical and cellular gradients from high-nutrient, low-waste regions containing viable, proliferating cells to low-nutrient, high-waste regions with arrested and dead cells.

FIG. 2 illustrates a diagram of microenvironmental simulation device highlighting spatial correlation of biochemical, cellular and metabolic signals (S) and direct connectivity to a mass transport model. Initial conditions for a given size device (critically: input metabolite concentrations, cell concentration and perfusion rate) quickly establish metabolite and catabolite gradients across the long axis of the device (simulated plots) due to cellular metabolism. The dynamics of these gradients are measured either in real time (input/output sensors, in situ nanosensors, direct optical analysis) or at discrete times after termination of the experiment and physical separation of cells/supernatants from known locations. Fitting of biochemical gradient data to mass transport model is used to extract metabolic rates as a function of location along the major axis.

FIG. 3 illustrates a cylindrical, transparent chamber is fitted with a conical output containing a retaining screen. Gel spheres containing nanosensors but no cells form a metabolically inert buffer zone. NanoCEMS fill the bulk of the cylinder, with another layer of inert spheres on top. A screen attached to a plunger is placed on the top, and a conical input that allows access to the plunger is added. The assembled device is then connected to a perfusion and external monitoring system. This simple process establishes a cylindrical packed bed of nanoCEMS that is perfused from one side. The inert buffer zones provide for a uniform flow profile as well as regions for calibrating the nanosensors. Device operation is initiated by single-pass perfusion with cell culture medium at 37°, which initiates cellular metabolism and the establishment of microenvironmental gradients. The biochemical gradients are continuously monitored using an external fluorescence microscope that is scanned along the major axis. The device is operated in continuous monitoring mode until a desired experimental endpoint. At the end of an experiment, the nanoCEMS are harvested by depressing the plunger while perfusion continues, ensuring maintenance of local in situ conditions during extrusion. Harvested nanoCEMS from known locations are separated from the medium by filtration, then either directly extracted for biochemical assay or the alginate is dissolved to release the cells for physiological assay.

FIG. 4 illustrates top phase contrast images show ˜100 μm alginate spheres created with two different cell concentrations. The bottom confocal fluorescence images show GFP-expressing tumor cells growing in CEMS.

FIG. 5A illustrates an embodiment of the new tissue/tumor model including a diagram of a simplified tissue region in vivo, with nutrients supplied by the vasculature. Extracellular biochemical gradients are created by diffusion in the face of cellular metabolite consumption (and catabolite production), creating cellular microenvironments defined by regions of: (1) proliferation, (2) quiescence and (3) necrosis.

FIG. 5B illustrates a diagram of a multicellular spheroid showing biochemical gradients and induced cellular pathophysiology. The major disadvantage to these systems is the inability to make spatially-correlated measurements of biochemical, metabolic and cellular microenvironments.

FIG. 5C illustrates a diagram of new model system in which diffusive supply is replaced by perfusion and the physical size of the device is expanded, allowing spatially-correlated gradient measurements.

FIG. 6A illustrates a technique for measuring pH gradients within the device including a carbodiimide chemical reaction used to couple fluoresceinamine to G monomers of sodium alginate, which can then be gelled using a crosslinking buffer.

FIG. 6B illustrates a pH calibration curve of the labeled sodium alginate, generated by adding increasing amounts of NaOH to increase the pH (triangles) and increasing amounts of HCl to decrease the pH (squares). Line illustrates a linear quenching over the pH range of interest for tumor microenvironments.

FIG. 6C illustrates a packed-CAM perfusion column prototype, showing the generation of a pH gradient by cellular metabolism that is externally measured with a fluorescence microscope scanned along the major axis.

FIG. 7 illustrates controlled formation of CEMS containing tumor cells. The top three panels show confocal fluorescence photomicrographs of GFP-expressing human colon tumor cells suspended within alginate droplets, including a single slice image (left) and a 3D stacked image (center) of a CEMS containing spatially-separated individual cells immediately following gelation, as well as a 3D image of cells growing into colonies within the CEMS. The bottom panels show phase-contrast photomicrographs demonstrating the generation of CEMS with different initial numbers of cells (50 cells/CEMS ˜1/3 tissue cellularity), as well as the proliferation and aggregation of cells within a single CEMS to form a multicellular spheroid after extended culture.

FIG. 8A illustrates the mathematical model to be used for analyzing the gradient data obtained from the device. The left panel shows how nutrient and waste concentration gradients in the extracellular space are fit using a 1D diffusion/consumption equation (top) with the effective perfusion rate replacing diffusion, while biochemical conditions inside the CEMS are modeled based on diffusive supply (middle). This model will be extended to incorporate measurements of cellular physiology (bottom).

FIG. 8B illustrates simulated oxygen concentration gradients across the device, illustrating the effects of altering the: a) perfusion rate, b) mean oxygen consumption rate and c) incorporating an oxygen consumption that varies along the major axis due to metabolic adaptation. Under defined conditions (perfusion rate, cell concentration), fits of this model to the measured biochemical gradients will be used to extract metabolic rates as a function of position in the device.

FIG. 9A illustrates morphological and chemical gradients found in tumor tissue (in vivo) and tumor spheroid (in vitro) sections. Gradients indicate characteristic spatially correlated regions as follows: proliferation/O2 60-80 mm Hg/pHe 7.4 quiescence/O2 30-60 mm Hg/ pHe 7.0 necrosis/O2 0-30 mm Hg/pHe 6.5.

FIG. 9B illustrates O2 and pHe nanosensors are crosslinked into microdroplets that encapsulate HIF-la wild-type (WT) or knockout (KO) cells. Left half of the sub-nano proposed mechanistic modulation of HIF-1 a in combined O2 and pHe microenvironments shows the degradation of HIF-1a in the cytoplasm and maintenance of aerobic metabolism; right shows the translocation of HIF-1a to the nucleus in the absence of O2, upregulation of survival proteins, and switch in metabolism to anaerobic glycolysis. 5×10⁶ cell encapsulating and sensing droplets are packed into the aforementioned chamber. An integrated optical analysis system measures microenvironmental parameters along the length of the perfusion instrument over several hours. Discrete recovery of the droplets, cells, and media supernatant at the end of an experiment allows for downstream analysis and correlation of HIF-1α phenotype to the measured microenvironmental conditions.

FIG. 10A illustrates results from the growth curve and membrane fluorescence dilution experiments. Left and Center show the monoculture growth curves (Top) and fluorescence decay (Bottom) for HIF-1α WT and KO cells treated with membrane dye (green or red) and unstained (purple). Right Top shows the average doubling time (DT) of the cells grown in a 50/50 co-culture (combined measurement (black) and unstained (purple)) and Bottom shows the independent fluorescence decay times (DecayT) with unstained (purple) and auto-fluorescence controls (blue).

FIG. 10B illustrates confocal and bright-field and microscopy of WT and KO cells at 6 hours and 72 hours of growth, respectively.

FIG. 11A illustrates an approach to high-throughput generation of nanoCEMS including a diagram of the nested capillary micro-nozzle system. Alginate pre-gel labeled with fluorescein is injected through an inner capillary and hydrodynamically-focused by an outer sheath containing a calcium crosslinking buffer. An external piezoelectric transducer vibrates the nozzles, resulting in formation of uniform pre-gel droplets surrounded by crosslinking buffer. Solidification of the pre-gel occurs within the droplets in air, ensuring a spherical shape prior to impacting a larger buffer reservoir.

FIG. 11B illustrates generation of uniform droplets of the viscous pre-gel solution from a single nozzle device (A and B) as well as a fluorescence photomicrograph of an inner fluorescent pre-gel solution surrounded by a crosslinking sheath (C). Note that the device is vibrated at 10-20 kHz, generating 10,000-20,000 CEMS per second (enough to fill a 5 cm×1 cm×1 cm device in approximately 10 minutes).

FIG. 12A illustrates at top left solution chemistries for the generation of water/water emulsions, at top right bright-field image of droplets formed from bulk emulsion stabilized for 24 hours, at bottom left a mathematical model used to determine the Reynolds number and flow rates for laminar flow profiles, and at bottom right results from measuring the kinematic viscosity of the dispersed phase, showing a 30× greater viscosity than water.

FIG. 12B illustrates at top a diagram of the droplet generating instrument showing integration of fluidics, electronics, and optics, at bottom left an image of a single channel device, and at bottom right bright-field images of droplet morphology.

FIG. 12C illustrates at top a picture of a two channel glass capillary assembly, at middle an optical light path for the integrated fluorescence imaging used to visualize hydrodynamic focusing in the core-annular flow, at bottom left fluorescence image of a two channel device with 10 μm fluorescein in the core flow, and at bottom right droplets in air vs. collected in slurry.

FIG. 13A illustrates a pH response curve from proof of principle experiment, demonstrating alginate functionalization and responsiveness in the physiological range.

FIG. 13B illustrates the perfusion rate or changing consumption rates as cells undergo proliferation and death influences the gradient distribution along the x-axis of the perfusion device.

FIG. 13C illustrates simulated oxygen gradients and metabolic activities in the device when varying (top) perfusion flow rates and (bottom) mean oxygen consumption rates. Mathematical model parameters are set from EMT6 spheroid cultures.

FIG. 14 illustrates a proposed mechanistic modulation of HIF-1α in combined pHₑ and O₂ microenvironments. Left half of the HKO3-TR^{WT} cell shows the degradation of HIF-1α in the cytoplasm and maintenance of aerobic metabolism. The right half of the cell shows the translocation of HIF-1α to the nucleus in the absence of O₂ and upregulation of survival proteins (solid line). The dotted line indicates the hypothetical degradation of HIF-1α when combinatorial acidic and hypoxic environmental conditions exist.

### DETAILED DESCRIPTION OF EMBODIMENTS OF THE INVENTION

Applicants disclose herein a system for examining biological cells in a new and novel 3-dimensional microenvironment. The system comprises a 3D microenvironmental perfusion device to obtain the parameters that are necessary to accurately capture the behavior of the biological system being investigated with mathematical modeling. This integration of engineering, physics and biology has the potential to significantly advance cancer research by providing an avenue to more accurately understand the relations of metabolic rates and phenotypic properties of cancer cells with microenvironmental factors, and thus has the potential to provide insight into the outcome of cancer treatment.

The disclosure provides for methods and systems in which many microenvironmental, physiological, metabolic and functional genomics parameters can be measured in a spatially- and temporally-correlated manner. This system will have numerous applications to research in basic tumor biology including, but not limited to, investigating the mechanistic basis for the microenvironmental regulation of tumor cell proliferation as well as to investigate the effects of transient nutrient deprivation, a notably neglected field. Other tumor biology applications include studies of the effects of the tumor microenvironment on cell metabolism, the regulation of gene/protein expression, and neoplastic progression. This system will be useful in a wide variety of other basic and applied cancer research areas, including drug development, radiobiology, and non-invasive diagnosis. The system is also potentially very useful for applied research on cell culture systems for biomaterial production and artificial organs.

As described herein, a perfusion chamber generally consists of a cylindrical chamber containing cells perfused within a 3D matrix. The perfusion chamber contains optical nanosensors incorporated within cell-encapsulated microgel spheres (nanoCEMS): uniform spherical beads containing cells and nanosensors within a biocompatible matrix. Similar to a chemical analysis bead-based column, this will allow uniform perfusion, controlled nutrient supply, in situ optical analysis and simple recovery of the cells as a function of location within the cylinder. The culture is perfused from one end, such that cellular metabolism establishes chemical gradients in nutrients and waste products across the cylinder, effectively reducing it to a “1D” device. The cells will, in turn, respond to their local biochemical environment in a myriad of ways, including alterations in metabolism, physiology, proliferation, viability, gene/protein expression and response to therapies. This device will be the first such system to allow detailed, precise and spatially-correlated measurements of complex chemical and cellular microenvironmental gradients. There are several key features of the system that directly drive successful implementation, validation and application, as explained below.

There are two types of signals (S) obtained directly from the device: measurements of the chemical microenvironment and assays of the cellular microenvironment. These will exist as spatially-correlated gradients along the long axis of the cylinder. One of the advantages of the design is that user can measure an essentially infinite number of discrete gradients through combinations of sensing technologies and assay methods applied. In addition to these experimentally-measured signals, integration of the data into a mathematical model will allow the extraction of metabolic parameters as a third type of “signal”. For example, from an oxygen concentration profile measured under known conditions of initial [0₂], perfusion rate and cell concentration, the spatial gradient in cellular oxygen consumption rates can be extracted. The types of metabolic parameters that could be extracted from the system are limited only by the experimental signals measured and manipulation of initial conditions.

At least three types of experimental measurements can be obtained for both the chemical and cellular microenvironments within the device. First, fluorescent nanosensors can measure a range of chemical signals. By embedding the sensors within an extracellular matrix sphere or having them internalized within the cells, the user can assay chemical gradients in both the extra- and intracellular microenvironments. Fluorescent signals from these sensors can be collected from discrete volumes within the device using an external microscope system that scans along the major axis of the transparent cylinder, producing signal gradients. Second, direct optical analysis can be used to collect elastic and Raman scattering as well as fluorescence signals from within the device. Elastic scattering signals can be used to measure cell number and viability, while Raman scatter spectra can extract concentrations of discrete chemical species. In some embodiments, it may be advantageous to use multiple excitation sources, allowing direct measurement of fluorescence that can be used to assay traditional chemical sensors bound to the matrix and cellular parameters (e.g. redox potential, fluorescent protein expression, mitochondrial activity, drug uptake). The third type of assay is different for the chemical and cellular signals. Measurements of the total change in chemical signal across the device (AS) will provide the average metabolic activity within the device. For example, Δ[O₂] will provide the average oxygen consumption rate within the device, important in and of itself but also useful as an independent calibration for the gradient measurements. Detailed assay of the cellular microenvironment can be obtained by extruding the culture from the device in a way that maintains the spatial distribution, then either recovering the cells for physiological assay by degrading the matrix or directly extracting metabolites, DNA, RNA and proteins.

Another key feature is the user has a very high degree of control over the initial conditions (S0) of the system. Physical parameters (chamber dimensions, flow rates, bead dimensions) are all adjustable based on the conditions desired. It is important to note that biochemical supply to cells in the device is due to perfusion, not diffusion, providing a much greater degree of control over microenvironmental conditions. Initial cellular concentrations and compositions, including the use of co-cultures, is easily adjustable. Since the cells are embedded in a biocompatible matrix, there are essentially no restrictions on the types of cells that can be employed. Importantly, use of mutant cell lines greatly expands the ability to manipulate the microenvironment. The input concentration of any biochemical is known and easily manipulated. Similarly, metabolic inhibitors, gene inducers and drugs can be introduced in a controlled fashion. In sum, the initial conditions of a given experiment can be manipulated over a very wide range, allowing production of correlated chemical and cellular microenvironments designed to address a great number of basic and applied research questions.

A further key feature is the dynamic range of the system. Several AS chemical measurements ([O₂], pH, [CO₂]) are real-time, employing continuous input/output sensors. The nanosensor and direct optical measurements are quasi-real-time, being limited only by the time required to scan across the cylinder (depending on the optical signal, estimated to be from seconds to minutes). This will allow relatively rapid kinetic analysis of chemical, cellular and metabolic microenvironments. Cell harvesting and extraction are destructive assays, thus can only be done at discrete times. However, the basic culture system is simple enough for easy replication, and the in/out and scanning optical analysis systems are designed to allow facile sampling of multiple replicate culture devices. Thus, the user can design experiments to measure the dynamics of microenvironmental regulation over a range covering relatively rapid metabolic alterations to changes induced by cell proliferation, differentiation and death. The availability of this dynamic range allows for a very wide range of applications, e.g., establishing a tumor cell culture within the device and allow it to “evolve” over a long period, monitoring the adaptation of the cells to the dynamic microenvironment.

The design of the physical device allows for directly coupled mathematical modeling of the microenvironmental conditions within the device. Extraction of metabolic parameters from signal gradients will be powerful tool, but it is also relatively simple in the“1D” device. Critically, the data produced by this device, for the first time, will allows development, validation and refinement of much more sophisticated models of the cellular response to dynamic, multifactorial microenvironments. Hypotheses derived from the mathematical models will also be very specific in terms of manipulation of the physical device, providing direct experimental testing of predictions. Again by example, the relatively simple experiment described above of allowing a tumor cell culture to “evolve” in the device over time will provide a wealth of data for testing and further development of our existing models of the regulation of tumor cell proliferation, metabolism and viability. In addition, combination of the device with a set of genetically engineered cells can be used to examine the role of a specific protein in the cellular response to microenvironmental stress. An example of such an experiment is to test the role of hypoxia-inducible factor 1 (HIF-1) in maintaining cell metabolism and viability in a stressful microenvironment is presented at the end of this section. Finally, the device may be used to test the effects of added drugs (e.g. cancer chemotherapy agents) on the metabolism, proliferation and viability of cells in different microenvironments.

### Perfusion Device Design and Operation

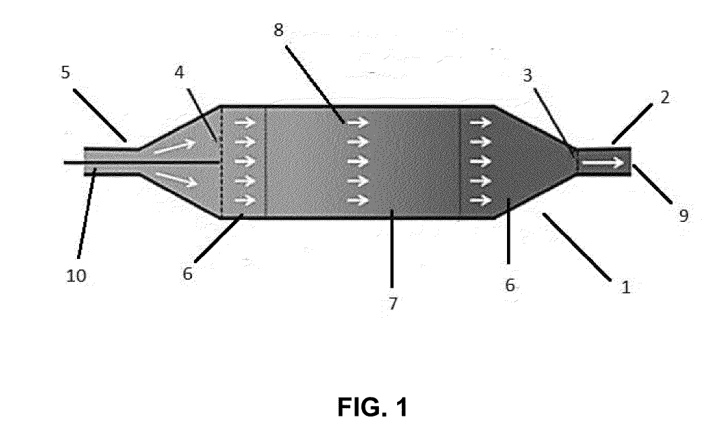

An embodiment of the perfusion chamber device is illustrated in FIG. 1. A profusion device 1 generally comprises a cylindrical, transparent chamber 8 fitted with a conical input 5 and a conical output 2, and a removable retaining screen 3 being situated near the conical output 2. The input and output is not required to be conical, but is preferred. Fluid can be perfused from the entrance opening 10 through the chamber body 8 and flows out the exit 9 to establish gradients within the device. Generally, inert gel spheres 6 containing nanosensors but no cells are introduced to fill the bottom conical volume, forming a metabolically inert buffer zone. CEMS, NanoCEMS or a combination thereof can be added to the chamber 8 to produce a packed bed of nanoCEMS 7, followed by a top layer of inert spheres 6 equal to the volume of the inert spheres of the conical output region. A screen/plunger 4 is positioned on top of the layer of inert spheres where the conical input 5 and entrance opening 10 allows external access to the plunger. The screen and plunger is preferably the same width as the inner diameter of the chamber. By depressing the screen/plunger assembly 4, the user is able to extrude the contents of the chamber through the exit 9 for analysis after removal of the retaining screen 3. The content of the chamber is extruded in a linear fashion, thereby maintaining the structural positioning of the material outside of the chamber as it positioned inside the chamber. In this way, discreet parts of the chamber can be targeted for further analysis such as DNA/RNA extraction, protein analysis, PCR including, gene expression analysis (RT-PCR, RT-qPCR), genetic fingerprinting, sequencing, ELISA, mass spectroscopy, DNA/RNA and protein blotting, and other analysis available to a person of ordinary skill in the art.

The device is preferably constructed of a transparent plastic, polymer or glass. It is important that the device be transparent such that visualization and detection of the nanoCEMS is easily accomplished by the end user. Preferably, the device is constructed of a biocompatible and impermeable to gas. In some embodiments, the device is constructed of glass or Lucite.

In some embodiments, the perfusion device has at least one flat side along the long axis. In other embodiments, the perfusion device has at least 2, or at least or at least 4 flat sides. In other embodiments, the perfusion device is square in shape along the long axis. A flat side can facilitate observation, for example, in microscopic observation or flow cytometry.

The assembled device can be connected to a perfusion and external monitoring system (described below). This process establishes a cylindrical packed bed of nanoCEMS that can be perfused from one side. The inert buffer zone near the input dampens turbulence to provide a uniform flow profile across the nanoCEMS bed. The upper and lower inert buffer zones have additional advantages for both calibration and harvesting, as explained below. The device is preferably assembled at 2°-4°, ensuring stable and reproducible initial conditions as well as the ability to assemble multiple devices whose operation can be initiated at the same time. Device operation is generally initiated by single-pass perfusion with cell culture medium of a defined composition at 37°, which initiates cellular metabolism and the initial establishment of microenvironmental gradients. The device is then perfused and monitored continuously for a period of time, followed by harvesting of the nanoCEMS from known locations after extrusion from the chamber if desired. In some embodiments, a pump such as a tubing pump, syringe pump or similar device is used to perfuse fluids through the perfusion chamber.

The perfusion and external monitoring system comprise, in some embodiments, a perfusion input and output equipped with in-line sensors for continuous monitoring of medium composition. In-line sensors, such electrodes for O₂ and CO₂ can be used, although a variety of optical or electrochemical sensors are available for specific applications. As used herein, “electrode” generally includes a composition, which, when connected to an electronic device, is able to sense a current or charge and convert it to a signal. Alternatively, an electrode can be a composition which can apply a potential to and/or pass electrons to or from connected devices.

Various electrodes for use with the device include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂0₆), tungsten oxide (W0₃) and ruthenium oxides; and carbon (including glassy carbon electrodes, graphite and carbon paste).

In some embodiments, in may be advantageous to measure a sample of the perfusion media (via an input sensor) to calculate initial compound concentrations, pH etc. before perfusion. Sampling of the perfusion media at the output side (via an output sensor) allows for off-line analysis of biochemicals in the spent medium (In/Out Assays). The difference in signals between the input and output sensors (ΔS) provides important data for calibration as well as for deriving mean rates of metabolic and catabolic processes in the culture.

The device in operation can be placed into a commercial temperature-regulating incubation chamber that is mounted on a precision translating microscope stage, which allows for collection of optical signals from different locations in the cylinder during operation. Optical signals are collected by an epi-fluorescence microscope or other optical system that collects light from a defined volume within the chamber. This external scanning optical sensing system is configured so that the perfusion device can be easily inserted and removed, allowing for long-term repeated measurements on a single device and sequential measurements on multiple devices. The inert buffer zones on either side of the nanoCEMS culture contain the same nanosensors but no cells, providing a defined region in which there are no gradients: optical signals measured in these two zones can be precisely calibrated by reference to the independent input/output sensors and biochemical analysis

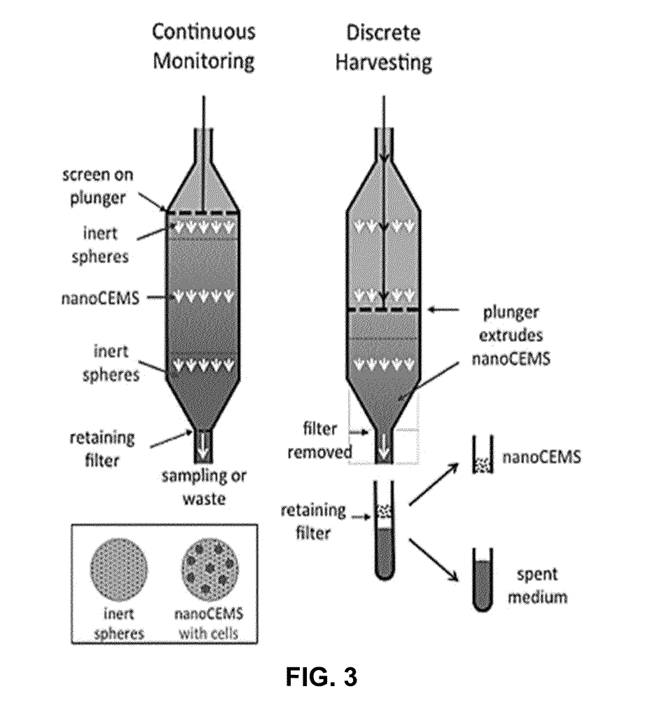

In some embodiments, it may be desirable to preserve both cellular and biochemical gradients within the nanoCEMS during harvesting, allowing the rapid recovery of CEMS from known locations within the device while maintaining the in situ microenvironmental gradients, as illustrated in FIG. 3. At the time of harvest, the lower retaining screen is removed and the plunger is precisely advanced into the chamber, eluting the nanoCEMS from the bottom of the device. Perfusion of the culture continues during harvesting, which not only assists with well-controlled removal of the nanoCEMS as a function of location, but also maintains the biochemical microenvironment around the nanoCEMS during extrusion. As described above, there is a buffer zone of inert (no cells) microspheres on either side of the packed bed of nanoCEMS: the upper inert zone allows complete removal of the nanoCEMS while the lower zone allows for establishment of uniform extrusion prior to sampling nanoCEMS. The extruded material is delivered into filter tubes that separate the nanoCEMS from the surrounding spent medium. Importantly, the perfusion system is running during the extrusion process (with an adjustment in flow rate to compensate for the rate of extrusion). This ensures that the biochemical microenvironment is maintained during nanoCEM recovery, since this is determined by the metabolism of the upstream cells that remain in the chamber. Collection of set volumes in sequential tubes results in recovery of both nanoCEMS and the biochemical microenvironment as a function of location within the device. The recovered nanoCEMS can be very rapidly frozen or directly subjected to chemical extraction, ensuring analysis of gene and protein expression profiles that have been minimally affected by the harvesting process. As described below, the nanoCEMS can also be disaggregated to yield cell suspensions from known locations. Volumetric sampling allows both cellular and extraction analysis of each recovered nanoCEMS sample. The separated spent medium can be assayed by standard molecular and biochemical methods to directly measure biochemical gradients.

A key feature of the proposed system is the very high degree of control over the initial conditions (So) of the perfusion device. For example, the physical dimensions of the device: the length and width of the chamber can be customized to a particular application. Various factors should be considered for operational devices, including the requirements and sensitivity of the scanning optical system, the ability to produce uniform flow with minimal wall effects, and the number of cells required for the cellular and extraction assays, as well as cell type. The gradients generated in a given device will depend primarily on cell concentration in the nanoCEMS, the perfusion rate and the initial medium composition. Embodiments of the device can be about 0.5-3 cm in diameter, about 1-2.5 cm in diameter, and more preferably about 1-2 cm in diameter. In other embodiments, the device is between about 2-15 cm long, about 4-12 cm long and most preferably about 5-10 cm long. NanoCEMS are generally about 100 μm in diameter containing ˜5 cells and a perfusion rate of ˜5 ml/min. In some embodiments, the mean particle size of the nanoCEMS can have a diameter between about less than 1 μm to about greater than 1 mm, preferably about 100 μm to about 300 μm, more preferably from about 100 to about 200 μm, and most preferably, about 100 μm in diameter. The size of the nanoCEMS is largely dictated by minimizing chemical and cellular gradients within the sphere so that the microenvironment within a given measured/harvested volume of the cylinder is uniform. The general procedure for operating the device establishes the initial operating conditions, then follows the evolution of gradients as a function of time. Importantly, the user can alter the flow rate (e.g. increasing it to compensate for cell proliferation) and medium composition (e.g. altering a specific metabolite or adding a drug) at various times during operation.

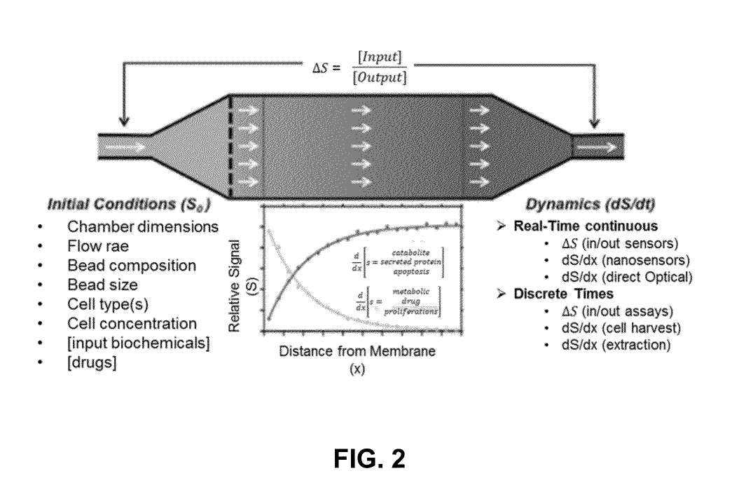

In some embodiments, and as shown in FIG. 2, a user can examine various chemical and cellular microenvironments. The chemical microenvironment (e.g. chemical gradients and a cell's response thereto) can be measured using a combination of nanosensors, direct optical measurements and through the changes in concentrations of specific parameters from the beginning of the chamber as compared to the end of the chamber (In/Out assay). Nanosensors can measure, for instance, the presence, absence or changes in concentration of O₂, pH, CO₂, secreted proteins, secreted proteins in response to a stimulus (e.g. exposure to a biological molecule or compound) and specific ligands in the device. Other molecules can be detected through direct optical measurements including glucose, lactate, glutamine, fatty acids and their derivatives, other metabolites and various drugs of interest such as chemotherapeutics, metabolic inhibitors, gene induction compounds (e.g. transcription factors commonly found in glucose metabolism or oncogene regulation) or the like. It is also possible, through In/Out assays, to measure O₂, pH, CO₂, glucose, lactate, glutamine, other metabolites, drugs and secreted proteins. In some embodiments, either nanosensors, direct optical observation or In/Out assays are used to measure a specific parameter. In other embodiments, any combination of nanosensors, direct optical observation and In/Out assays are used to measure a specific parameter(s).

In further embodiments, the cellular microenvironment can be measured. Similar to measurements of the chemical microenvironment, measurements of the cellular microenvironment can include nanosensors, direct optical observation and harvesting of nanoCEMS. Using nanosensors, a user can measure intracellular O2, intracellular pH and various metabolic parameters of interest. Through direct optical observation, a user can measure, for example, cell number, cell proliferation, mitochondrial activity, cell viability, Fp reporter, drug uptake and other metabolic parameters. A user can also, through actuation of the plunger/screen, extrude the contents of the chamber for further analysis through harvesting of the nanoCEMS. Harvesting can be used to measure cell number, cell volume, cell proliferation, mitochondrial activity, cell viability, cell apoptosis, metabolic parameters, protein expression, gene expression, and therapy survival (e.g. drug response).

Accordingly, in some embodiments, a chemical/cellular microenvironment parameter such as pH, O2 or CO2 concentration is measured in the perfusion media prior to perfusion via a nanosensor (such as an electrode). A molecule of interest is added to the perfusion media and perfused through the chamber to establish a gradient of the molecule of interest under constant perfusion. After a certain time, the cells in the nanoCEMS establish their own gradients (nutrient/waste product, secreted proteins or other solutes) in response to the molecule of interest. These gradients can be measured in discreet locations about the device, as well as at the fluid output, through optical detection or other means as described herein. The nanoCEMS may also be harvested to further examine the cellular and/or chemical microenvironment.

In other embodiments, the chemical/cellular microenvironment is measured at discreet locations within the device prior to the addition of a molecule of interest.

In further embodiments, a chemical/cellular microenvironment parameter such as pH, O2 or CO2 concentration is measured in the perfusion media prior to perfusion via a nanosensor (such as an electrode) and after the perfusion media exits the perfusion device.

### NanoCEM Fabrication, Characterization and Dissociation

In some embodiments, CEMS and nanoCEMS are fabricated using encapsulation of both nanosensors and cells within gel microspheres composed of a biocompatible matrix material that is preferably optically transparent, easily solidified and subsequently dissolved under conditions that maintain cell viability, and amenable to modifications to better mimic the extracellular matrix in tissues/tumors.

Currently, no methods for rapidly producing large numbers of cell-embedded alginate spheres ˜100 μm in diameter exist (e.g. ˜4×10⁶ nanoCEMS are needed to fill a device 1 cm wide×5 cm long). Here, Applicant provides a system and method for producing such microspheres.

The system and method for producing spheres generally comprises, for example, a simple flow cell with an inner 70 μm nozzle aligned within an outer 250 μm nozzle. Such a flow cell can be created using pulled glass pipettes or other suitable material. A polymer matrix pre-gel solution, or other polymer, biopolymer, or ECM components as described below, containing known concentrations of cells and nanosensors is input to the inner pipette and a calcium polymerizing solution, or other suitable ionic or non-ionic crosslinker, is input to the outer pipette. The flow cell creates a hydrodynamically-focused inner pre-gel jet coaxially-aligned within a larger jet of calcium solution that exits the larger nozzle into air. A piezoelectric transducer is attached directly to the outer pipette, producing vibrations that drive the generation of precise droplets. The function generator driving the PZT also flashes an inexpensive LED array at the same frequency, enabling freeze-frame imaging of droplet formation with a simple digital movie camera or other imaging device. Pre-gel and calcium concentrations are controlled so that polymerization does not occur prior to droplet formation (˜10 μsec after the streams contact), so the droplets contain an inner pre-gel sphere within an outer droplet of calcium solution. The outer surface of the pre-gel sphere hardens during transit in air (˜0.5 sec) to a receiving solution, so that the resulting nanoCEMS maintain their spherical shape and do not aggregate upon entering the receiving solution. In some embodiments, the outer droplet further comprises a surfactant or stabilizing agent such as dextran or Pluronic F-127 such that the outer droplet forms an outer shell over the polymerized inner core. A device with a 250 μm exit nozzle generates droplets in the range of 2-10 kHz depending on the flow rates, generating uniform nanoCEMS at rates up to 10,000 per second (e.g. ˜7 minutes to generate enough nanoCEMS to fill a 1×5 cm device). The components for these droplet generating flow cells are low cost so that production rates could be further increased, if needed, by running several flow cells simultaneously.

The present disclosure also provides for a system for fabricating microspheres. In some embodiments, the system comprises one or more reservoirs containing separate mixtures of a polymer solution and a crosslinking solution. The polymer mixture can comprise, for example, a polymer, biopolymer, a biological cell, nanosensor, ECM component or any combination thereof. The crosslinking mixture can comprise, for example, an ionic crosslinking agent and a stabilizing agent. The mixtures are separately pumped into a dispensing device having at least one outer chamber with an outer dispensing nozzle and at least one inner chamber with an inner dispensing nozzle such that the inner chamber and dispensing nozzle are nested within the outer chamber and outer dispensing nozzle. The mixtures flow though the separate inner and outer chambers where they are mixed upon exiting the nozzles. A piezoelectric transducer is coupled to the dispensing device and determines the size of the mixture droplets through acoustic modulation of the mixture stream, thereby determining the breaking rate of the dispersed stream into droplets. The droplets are crosslinked in the air and are captured in a receiving solution positioned beneath the dispensing device. The fabrication of the microsphere droplets can be monitored through a high speed camera positioned to view the droplets as they exit the dispensing device. The camera with an appropriate lens/filter is capable of capturing images at the same rate the droplets are formed. A light source is positioned opposite the camera to assist in visualizing the droplets. In some embodiments, the light source is a Light Emitting Diode (LED) and diffusion array where the LED emits light at about the 470 nm wavelength and the light waves may be focused using collimating optics and lenses to illuminate and visualize the droplets. The system also comprises a function generator that is coupled to an amplifier, the amplifier being further coupled to a power supply, the piezoelectric transducer and an oscilloscope. The function generator may also be coupled to the LED and diffusion array.

The CEMS and nanoCEMS may comprise at least one biocompatible, and/or biodegradable polymers. Examples of such polymeric materials can include a suitable hydrogel, hydrophilic polymer, hydrophobic polymer, bioabsorbable polymers, and monomers thereof. Examples of such polymers can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanhydrides, polyphosphazenes, poly(phosphoesters), polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, polyethylenes, polypropylenes, polyaliphatics, polyvinylalcohols, polyvinylacetates, hydrophobic/hydrophilic copolymers, alkylvinyl alcohol copolymers, ethylenevinyl alcohol copolymers (EVAL), propylenevinyl alcohol copolymers, polyvinylpyrrolidone (PVP), poly(L-lysine), poly(lactic acid-co-lysine), poly(lactic acid-graft-lysine), polyanhydrides (such as poly(fatty acid dimer), poly(fumaric acid), poly(sebacic acid), poly(carboxyphenoxy propane), poly(carboxyphenoxy hexane), poly(anhydride-co-imides), poly(amides), poly(iminocarbonates), poly(urethanes), poly(organophasphazenes), poly(phosphates), poly(ethylene vinyl acetate) and other acyl substituted cellulose acetates and derivatives thereof, poly(amino acids), poly(acrylates), polyacetals, poly(cyanoacrylates), poly(styrenes), poly(vinyl chloride), poly(vinyl fluoride), poly(vinyl imidazole), chlorosulfonated polyolefins, polyethylene oxide, combinations thereof, polymers having monomers thereof, or the like.

In other embodiments, the polymer may comprise a poly alkylene oxide. The term “poly alkylene oxide” as used herein refers to a class of compounds comprising at least two repeating units comprising an ether-alkyl group wherein the alkyl group forming the backbone of the repeating unit comprises from 2 to 3 carbon atoms which may be un-substituted or substituted. Non-limiting examples of applicable substituent groups include: hydroxyl, carboxylic acid, alkyl, and alkoxy wherein alkyl and alkoxy groups may be un-substituted or substituted with substituent groups such as hydroxyl and epoxy.

By way of example, the poly alkylene oxide compounds can include: polyethylene glycol, polyethylene glycol monoalkyl ethers, trimethylolpropane ethoxylate, pentaerythritol ethoxylate, and glycerol ethoxylate, polyethylene glycol, polyethylene glycol monoglycidyl ether, poly(ethylene glycol) 2-aminoethyl methyl ether, polyethylene glycol mono (2-aminoethyl)ether, polyethylene glycol diamine, polyethylene glycol bis(3-aminopropyl)ether, polyethylene glycol diglycidyl ether; polyethylene glycol bis(2-chloroethyl)ether, polyethylene glycol bis(2-bromoethyl)ether, polyethylene glycol 2-chloroethyl methyl ether, polyethylene glycol 2-bromoethyl methyl ether, sulfonate of polyethylene glycol methyl ether and a,w-disulfonate of polyethylene glycol, or any combination thereof.

The CEMS and nanoCEMS may also comprise, for example, biopolymers, including but are not limited to gelatin, calcium alginate, sodium alginate, collagen, oxidized regenerated cellulose, carboxymethylcellulose, hydroxypropyl cellulose, amino-modified cellulose, whey protein, chitosan, chitin, dextran sulfate, heparin, chondroitin sulfate, hyaluronic acid and combinations of any two or more thereof. In another embodiments, sodium or calcium alginate is used to create the CEMS and nanoCEMS.

In further embodiments, the CEMS and nanoCEMS may comprise a mixture of at least one polymer and at least one biopolymer as listed herein. In some embodiments, the polymer or biopolymer is selected from the group consisting of alginate, agarose, chitosan, cellulose, collagen, xantham, poly ethylene glycol, polyvinal alcohol, polyurethane, poly(ether sulfone), polypropylene, poly-L-lysine, poly-L-ornithine, poly(methylene-co-guanidine), sodium polystyrene sulfate, polyacrylate, or poly(acrylonitrile-sodium methallysulfonate, or any combination thereof.

In further embodiments, the CEMS and nanoCEMS are engineered to mimic the extracellular matrix (ECM). CEMS and nanoCEMS having an ECM component can further comprise proteoglycans (e.g. heparin sulfate, chondroitin sulfate, and keratin sulfate), non-proteoglycans polysaccharides (e.g. hyaluronic acid), fibers (e.g. collagen, elastin, fibronectin and lamin) and other molecules. These substances can be added to a polymer or biopolymer as listed herein to form a microsphere with an ECM component. In other embodiments, the CEMS and nanoCEMS are constructed of Matrigel® (Corning Life Sciences).

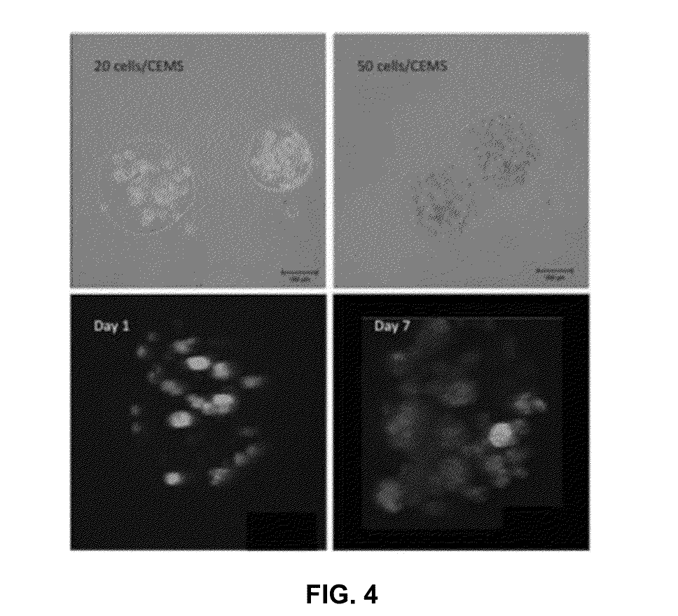

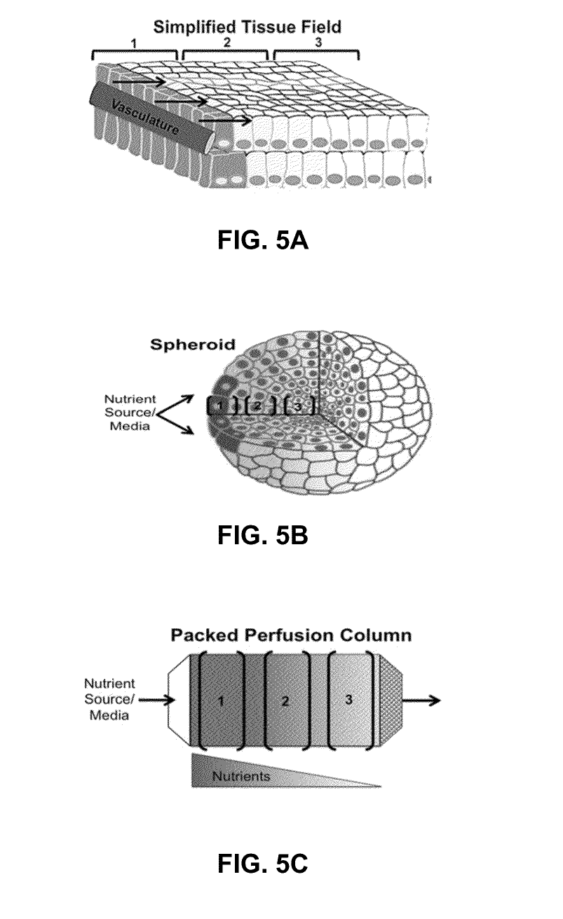

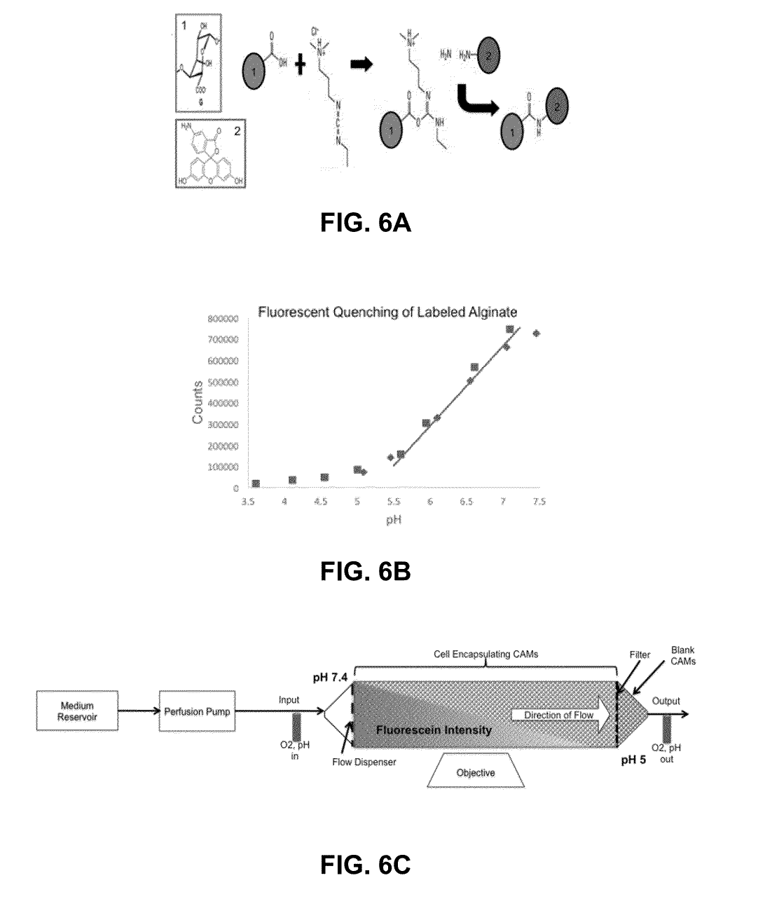

As shown in FIG. 4, Applicant has generated uniform CEMS over a wide range of cell concentrations, up to ˜1/3 the cell concentration in tissue. CEMS diameter can be controlled over a limited range (e.g. 70-150 μm using a 70 μm inner orifice) by modifying the relative flow rates; other sizes can be created using larger or smaller orifices. CEMS can be cultured for extended times (weeks) in suspension using standard cell culture media, without coming apart or aggregating. A variety of human and rodent tumor cells proliferate in the CEMS (FIGS. 5A, 5B, 5C). Interestingly, when the initial cell concentration is fairly low, the cells grow as minicolonies with intimate cell-cell contacts. Long-term culture of CEMS or use of a high initial cell concentration results in the formation of uniform cellular aggregates (multicellular spheroids). Thus, starting with an initial low cell concentration , the user can culture cells within CEMS for 4-5 cell divisions before the alginate encapsulation is lost, providing a large dynamic range for experiments using the device. In some embodiments, the CEMS are formed from Matrigel using an even simpler one-orifice nozzle: the gel droplets solidify directly by passing them through heated air. Alginate is more transparent than Matrigel and is therefore preferred. In further embodiments, ECM proteins and other ligands as listed herein ligands can be conjugated to, for example, alginate and adding various chemical groups to allow polymerization with the microspheres (Chou et al. Osteoarthritis Cartilage 17: 1377-1384 (2009)).

The cells used in the CEMS and nanoCEMS are preferably human cells, but can also be other Eukaryotic cells such those cells derived from an animal such as, but not limited to primates, rodents, felines, canines, poultry, ruminants, equine and swine. The cells can also be obtained from a biological sample taken from a subject, human or otherwise, having or suspected of having cells in a diseased state. In some instances, the disease state may be cancer.

As used herein, “obtained from a biological sample” or “obtaining a biological sample” refers to such methods as will be well known to the skilled worker. A biological sample may be obtained directly or indirectly from the subject. The term “obtaining” a biological sample may comprise receiving a biological sample from an agent acting on behalf of the subject. For example, receiving a biological sample from a doctor, nurse, hospital, medical center, etc., either directly or indirectly, e.g. via a courier or postal service.

In other examples, a sample containing cells, diseased cells, cancerous cells or suspected as containing diseased cells is obtained from the subject using a fine needle aspirate (FNA) sample. Methods of obtaining a FNA sample, processing and/or storage of such a sample are also well known to the skilled worker. In other examples, a sample is obtained from surgical dissection. In other embodiments, a physician prepares the samples or other qualified individual and provided for examination.

The term “sample” as used herein, encompasses a variety of cells, cell-containing bodily fluids and/or secretions as well as tissues including, but not limited to a cell(s), tissue, whole blood, blood-derived cells, plasma, serum, tumors, sputum, mucous, bodily discharge, and combinations thereof, and the like.

In some embodiments, the cell can be, for example, embryonic stem cells, amniocytes, blastocysts, morulas, and zygotes; leukocytes, e.g. peripheral blood leukocytes, spleen leukocytes, lymph node leukocytes, hybridoma cells, T cells (cytotoxic/suppressor, helper, memory, naive, and primed), B cells (memory and naive), monocytes, macrophages, granulocytes (basophils, eosinophils, and neutrophils), natural killer cells, natural suppressor cells, thymocytes, and dendritic cells; cells of the hematopoietic system, e.g. hematopoietic stem cells (CD34+), proerythroblasts, normoblasts, promyelocytes, reticulocytes, erythrocytes, pre-erythrocytes, myeloblasts, erythroblasts, megakaryocytes, B cell progenitors, T cell progenitors, thymocytes, macrophages, mast cells, and thrombocytes; stromal cells, e.g. adipocytes, fibroblasts, adventitial reticular cells, endothelial cells, undifferentiated mesenchymal cells, epithelial cells including squamous, cuboid, columnar, squamous keratinized, and squamous non-keratinized cells, and pericytes; cells of the skeleton and musculature, e.g. myocytes (heart, striated, and smooth), osteoblasts, osteoclasts, osteocytes, synoviocytes, chondroblasts, chondrocytes, endochondral fibroblasts, and perichonondrial fibroblasts; cells of the neural system, e.g. astrocytes (protoplasmic and fibrous), microglia, oligodendrocytes, and neurons; cells of the digestive tract, e.g. parietal, zymogenic, argentaffin cells of the duodenum, polypeptide-producing endocrine cells (APUD), islets of langerhans (alpha, beta, and delta), hepatocytes, and kupfer cells; cells of the skin, e.g. keratinocytes, langerhans, and melanocytes; cells of the pituitary and hypothalamus, e.g. somatotropic, mammotropic, gonadotropic, thyrotropic, corticotropin, and melanotropic cells; cells of the adrenals and other endocrine glands, e.g. thyroid cells (C cells and epithelial cells); adrenal cells; or a combination thereof. In some embodiments, a single three dimensional gel microenvironment may comprise at least two different types of cells. In some embodiments, CEMS and nanoCEMS may comprise 3, 4, 5, 10, or more types of cells.

The various types of cells that are used herein are grown and cultured according to methods well known in the art. Generally, a cell culture medium contains a buffer, salts, energy source, amino acids (e.g., natural amino acids, non-natural amino acids, etc.), vitamins, and/or trace elements. Cell culture media may optionally contain a variety of other ingredients, including but not limited to, carbon sources (e.g., natural sugars, non-natural sugars, etc.), cofactors, lipids, sugars, nucleosides, animal-derived components, hydrolysates, hormones, growth factors, surfactants, indicators, minerals, activators of specific enzymes, activators inhibitors of specific enzymes, enzymes, organics, and/or small molecule metabolites. These components may also be a molecule of interest where the effect of said molecule of interested is investigated using an embodiment of the disclosure.

In some embodiments, the cell media and/or perfusion media may comprise growth and differentiation factors including, but not limited to Acidic fibroblast growth factor, Adrenomedullin, Angiopoietin, Autocrine motility factor, Basic fibroblast growth factor, Bone morphogenetic proteins, Brain-derived neurotrophic factor, Cartilage-derived growth factor, Epidermal growth factor, erythropoietin, Fibroblast growth factor, Glial cell line-derived neurotrophic factor, Granulocyte colony-stimulating factor, Granulocyte macrophage colony-stimulating factor, Growth differentiation factor-9, Healing factor, Hepatocyte growth factor, Hepatoma-derived growth factor, Insulin-like growth factor, Keratinocyte growth factor, Migration-stimulating factor, Myostatin, Nerve growth factor (NGF) and other neurotrophins, Platelet-derived growth factor, Thrombopoietin, Transforming growth factor alpha, Transforming growth factor beta, Tumor necrosis factor-alpha, Vascular endothelial growth factor, placental growth factor, Bovine Somatotrophin, IL-1, IL-2, IL-3, IL-4-, IL-5, IL-6 and IL-7, or combinations thereof. These growth and differentiation factors may also be a molecule of interest where the effect of said molecule of interested is investigated using an embodiment of the disclosure.

In some embodiments, the cell used to create a CEMS or nanoCEMS may comprise normal cells, benign cells, cancer cells, immortalized cells, stem cells, genetically engineered cells and patient derived cells or a combination thereof. In a specific embodiment, the cells are cancerous cells.

In other aspects, various research important cells which can be encapsulated as a CEMS or nanoCEMS include, but are not limited to, Chinese hamster ovary (CHO) cells, HeLa cells, Madin-Darby canine kidney (MDCK) cells, baby hamster kidney (BHK cells), NSO cells, MCF-7 cells, MDA-MB-438 cells, U87 cells, A172 cells, HL60 cells, A549 cells, SP10 cells, DOX cells, DG44 cells, HEK 293 cells, SHSY5Y, Jurkat cells, BCP-1 cells, COS cells, Vero cells, GH3 cells, 9L cells, 3T3 cells, MC3T3 cells, C3H-10T1/2 cells, NIH-3T3 cells, and C6/36 cells.

In order to model transport within the device, it is important to know the transport properties of the nanoCEMS. Diffusion rates can be measured by various means including fluorescence recovery after photobleaching (FRAP) measurements on unperfused nanoCEMS equilibrated with inert fluorescent markers of various sizes. Photobleaching is the disappearance of fluorescent signal following the irreversible breakdown of fluorescent molecules after their interaction with molecular oxygen. The observation and measurement of FRAP allows scientists to investigate the diffusion and motion of biological molecules. This is particularly useful for studying the mobility of fluorescently labelled proteins. With FRAP, the fluorescently tagged protein of interest is visualized at low light intensity followed by photobleaching of a user-defined region of interest with high intensity light, causing the fluorophores to bleach in the selected region.

In other embodiments, a fluorophore can be added to the perfusion device, and the sample containing nanoCEMS is irradiated with an appropriate wavelength to initiate fluorescence, and the rate at which fluorescence returning to the previously irradiated area is observed and recorded.

Alginate spheres, for example, are porous, so convective transport within the nanoCEMS during perfusion is possible. However, the rate of transport within the nanoCEMS can be determined by confocal FRAP on individual nanoCEMS within a perfused device. FRAP measurements within a larger optical volume can be used to estimate the local flow rate and uniformity within the device during operation. FRAP measurements can be calibrated and validated using a simple model of flow in a packed-bed reactor and the bulk flow rate. Diffusion and flow within and between the nanoCEMS can be measured by adjusting the optical volume interrogated and switching the perfusion on and off, thereby providing a method to characterize basic transport parameters in different devices.

In some embodiments, at least two types of assays for analyzing nanoCEMS harvested from different regions of the device can be used. As shown in FIG. 3, nanoCEMS can be collected into filter tubes, allowing facile separation of the microspheres and the spent medium surrounding them through extrusion out of the chamber by use of the reciprocating plunger. Importantly, since the device is operation during harvesting, the spent medium recovered from different regions is the chemical microenvironment around the nanoCEMS at that location during operation. Biochemical composition (metabolites, proteins) of these spent medium samples will be determined using standard biochemical and molecular biology methods (Mourant et al., Biophysical journal 85: 1938-1947 (2003); LaRue et al., Cancer research 64: 1621-1631(2004); Freyer et al., In vitro cellular & developmental biology: journal of the Tissue Culture Association 25: 9-19 (1989)). The separated nanoCEMS can be rapidly washed and then extracted directly to yield metabolites, proteins, and nucleic acids from cells at different locations using techniques that are well known in the art. In some instances, at least some of the recovered nanoCEMS can be dissolved to yield suspensions of viable cells. For example, alginate-embedded cultures can be dissolved using calcium chelating agents and/or alginase. In other embodiments, cells can be recovered from a suspension of alginate CEMS by calcium chelation alone, probably owing to the high surface area to volume of the microspheres. As shown in FIG. 4, cells form cell-cell attachments during culture in CEMS, so some situations may require a combination of alginate dissolution with proteolytic digestion to obtain single cell suspensions.

### Nanosensors

There are many types of nanosensors available for use with the present disclosure. In some embodiments, the nanosensor is a fluorescent nanosensor (i.e. fluorophore). As used herein, the term “fluorophore” is meant to include a free molecule, or moiety of a larger molecule or conjugate that can be induced to emit fluorescence when irradiated, i.e., excited, by electromagnetic radiation of an appropriate wavelength. More particularly, a fluorophore can be a functional group of a molecule or conjugate that absorbs light of a certain wavelength and emits light at different wavelength. The intensity and the wavelength of the light emitted, as well as other fluorescence properties including, but not limited to, fluorescence lifetime, anisotropy, polarization, and combinations thereof, depend on the identity of the fluorophore and its chemical environment. A fluorophore can include a fluorescent molecule, such as a fluorescent dye.

Typically, ratiometric methods are employed when using fluorophores. Ratiometric methods are based on the use of a ratio between two fluorescence intensities. This allows correction of artifacts due to bleaching, changes in focus, variations in laser intensity, etc. but makes measurements and data processing more complicated.

Ratiometric indicators show a shift in their emission or excitation spectra when they bind to certain molecules, therefore they can be classified as dual emission or dual excitation indicators. Measurement of these compounds is achieved by using two excitation lasers (if they are dual excitation indicators) or two detection ranges (it they are dual emission indicators).

If a ratiometric indicator is used, intensity ratio is preferably calculated at wavelengths where the difference of fluorescence between bound and free indicator is maximum.

A ratiometric quantification can also be done using a mixture of an intensity shift indicator and an insensitive fluorescence compound (i.e. Fluo-3). Excitation and emission wavelengths of compounds listed herein are well known in the art.

In some embodiments, the fluorophore is a molecular dye probe which can be positioned inside a cell, or be positioned inside the nanoCEMS. Various molecular dye probes include, but are not limited to umbelliferone, fluorescein, fluorescein isothiocyanate, rhodamine, dichlorotriazinylamine fluorescein, tetramethylrhodmaine isothiocyanate, dansyl chloride, phycoerythrin, 8-Hydroxypyrene-1,3,6-Trisulfonic Acid, Lysosensor™, Oregon Green™, 9-Amino-6-chloro-2-Methoxyacridine, pHrodoTM, seminaphthorhodafluors (SNARF® dyes), carboxy-SNARFTM, carboxynapthofluorocein, fluorescein diacetate, 2′-7′-bis(carboxyethyl)-5(6)-carboxyfluorescein (BCECF). Fluorescein, for example, has an absorption maximum at 494 nm and emission maximum of 512 nm (in water).

In other embodiments, fluorophores can be conjugated to another molecule that can specifically bind to a molecule of interest, such as a receptor, whereby binding of the molecule of interest cause a detectable change in fluorescence emission when irradiated with an appropriate laser light wavelength.

Non-limiting examples of “receptor” can include an antibody, an antibody fragment, an aptamer, or an enzyme, or portions thereof.

The term “antibody” or “antibodies” as used herein refers to proteins used by the immune system to identify and/or neutralize foreign targets such as bacteria or viruses. Antibodies tend to be Y-shaped glycoproteins produced by B-cells and secreted by plasma cells. Antibodies recognize particular parts of a target known as antigens and bind to a specific epitope thereon. The fluorophore can be conjugated, for example, to an antibody.

A molecule (solutes) of interest may include, for example, amino acids, carbohydrates, nucleotides, nucleosides, hormones, organic acids, vitamins, lipids including neutral lipids, phospholipids, free fatty acids, total fatty acids, triglycerides, cholesterol esters, phosphatidylcholines, and phosphatidylethanolamines, naphthalenes, nucleosides, phenazines, quinolines, terpenoids, waste products, nutrients, proteins and other small peptides.

In other embodiments, fluorophores can be coated over at least a portion of a nanoparticle surface. Any fluorophore listed herein can be used to coat a nanoparticle such as is described herein and, for example, in U.S. Pat. No. 9,023,372 and US Pat. Pub. No. 20060213779. The coated nanoparticle can further be incorporated into the cell-embedded matrix spheres to form nanosensor-cell-embedded matrix spheres. In other embodiments, a fluorophore is situated within a nanoparticle.

A ‘nanoparticle’ as used herein is defined as a nanoscale structure having substantially similar dimensions of length, width and depth. For example, the shape of a nanoparticle may be a cylinder, a sphere, an ellipsoid, or a faceted sphere or ellipsoid, or a cube, an octahedron, a dodecahedron, or another polygon. In other examples, the nanoparticle may comprise a substantially irregular three-dimensional shape. The size of the nanoparticle may range from about 5 nm to about 200 nm, for example, in diameter or dimension. In some examples, the nanoparticle dimensions may be within a range of about 50 nm to about 100 nm, or about 25 nm to about 100 nm, or about 100 nm to about 200 nm, or about 10 nm to about 150 nm, or about 20 nm to about 200 nm.

Nanoparticles of the present disclosure can comprise, for example, silicate, zinc oxide, silicon dioxide, metals, metal oxides, polymers, fullerenes or composites thereof. In further embodiments, nanoparticles can comprise cadmium selenide, cadmium sulfide, silver sulfide, zinc sulfide, zinc selenide, lead sulfide, gallium arsenide, silicon, tin oxide, iron oxide, indium phosphide, tin dioxide, titanium dioxide, indium(III) oxide, palladium-tin dioxide-antimony, iron(III) oxide, zinc oxide, bismuth(III) oxide-molybdenum trioxide, and combinations thereof. In other embodiments, a metal nanoparticle is selected from the group consisting of gold, silver, platinum, palladium, iridium, rhodium, osmium, iron, copper, cobalt, and alloys thereof. In some embodiments, the nanoparticle is a silver, gold or silica nanoparticle. A non-limiting method for making nano-particles and integrating the nanoparticles into a gel matrix is disclosed in U.S. Publication 2003/0138490, which is incorporated by reference.

The invention also provides a nanosensor comprised of nanoparticles having a detection means attached thereto whereby the nanoparticles provide a detectable change in their UV-visible absorption spectrum in response to the binding of a molecule of interest. This change may be observed by instrument or by the naked eye. Metal nanoparticles are small enough to interact intimately with biological or chemical species. Such interaction is facilitated by their comparable size and by the large surface area to volume ratio of the nanoparticles. Molecular components can be readily attached to the nanoparticle surface. The attachment, which can be by nonspecific adsorption or interactions involving covalent or electrostatic bonding, affects the Surface Plasmon Resonance (SPR) of the nanoparticle and alters the spectral response. This alteration of the spectral response can be observed either as a wavelength shift in spectral peak, a diminishment or enhancement of the peak absorbance, or a combination of these. This sensitivity of the surface of these nanoparticles to the molecules in the surrounding environment makes them ideal for nanosensor applications.

Metal nanoparticles that differ in size, shape and composition scatter light of different wavelengths according to their distinct SPR. This is again due to the influence of these factors on the spectral response of the SPR. The most typical metal nanoparticle shape is spherical and these have a characteristic single SPR spectral peak. If a metal nanoparticle has a non-spherical shape, for example ovoid, then the SPR will exhibit more than one peak. This occurs as the nanoparticles are no longer isometric and the SPR electrons have more than one oscillation axis. In the case of ovoid nanoparticles, electronic oscillation about the major and minor axes will result in at least two peaks in the SPR spectrum. An advantage of non-isometric metal nanoparticles is their increased sensitivity, which in part arises from the presence of the additional SPR spectral peaks. Since the most energetically favorable nanoparticle morphology is spherical, the additional SPR peaks of non-spherical nanoparticles are therefore extra-sensitive to the local environment, and changes in the spectral profile are more easily observable than in the case of single SPR peak spherical metal nanoparticles.

The basic construction of these nanoparticle nanosensor includes a receptor (such as described herein) which interacts selectively with a target molecule and an indicator which generates a signal when an interaction has occurred. In some embodiments, the nanoparticles themselves can the indicator component. Any suitable recognition system may be used as the detector and target components. Additionally more than one type of receptor may be attached to the nanoparticles such that the sensor would be capable of detecting more than one target molecule simultaneously.

The receptor molecule may be attached to the nanoparticles via, for example, a thiol or amine group. This group may be part of the receptor molecule's chemical structure or may be introduced through the use of a linker molecule.

Larger molecules such as proteins (e.g. antibodies) may also be attached to nanoparticles. The nanoparticles of the invention are expected to have an overall negative charge. This charge can play a role in enabling large molecules such as proteins to bind to the surface. In the case of proteins a number of features including the net positive charge of a protein (lysine), together with hydrophobic binding (tryptophan) and sulphur bonding (cysteine and methionine) can facilitate the attachment between the nanoparticle and protein. This enables proteins to be readily adsorbed onto the nanoparticle surface. Proteins may also be coupled onto the nanoparticles. The use of tri-sodium citrate in preparing the nanoparticles means that carboxyl groups are present on the nanoparticle surface. This allows the use of well-known coupling methods such as carbodiimide coupling to attach proteins to the nanoparticles through a reaction which joins amino groups on the protein to carboxyl groups on the nanoparticles.

Quantum dots are fluorescent semiconductor nanocrystals having a characteristic spectral emission, which is tunable to a desired energy by selection of the particle size, size distribution and composition of the semiconductor nanocrystal. The emission spectra of a population of quantum dots have linewidths as narrow as 25-30 nm, depending on the size distribution heterogeneity of the sample population, and lineshapes that are symmetric, gaussian or nearly gaussian with an absence of a tailing region. Advantageously, the range of excitation wavelengths of the quantum dots is broad. Consequently, this allows the simultaneous excitation of varying populations of quantum dots in a system having distinct emission spectra with a single light source, e.g., in the ultraviolet or blue region of the spectrum.

Other nanosensor further include chromoionophores. A chromoionophore is an ionophore that changes its optical properties in the visible spectrum depending on the state of complexation. Chromoionophores for use in sensors are typically proton-sensitive dyes that change absorbance (and fluorescence in many cases) depending on the degree of protonation, although chromoionophores that change absorbance in response to other ions can also be used. Examples of suitable chromoionophores include Chromoionophore I (i.e., 9-(Diethylamino)-5-(octadecanoylimino)-5H-benzo[a]phenoxazine), Chromoionophore II (i.e., 9-Dimethylamino-5-[4-(16-butyl-2,14-dioxo-3,15-dioxaeicosyl)phenylimino]benzo[a]phenoxazine) and Chromoionophore III (i.e., 9-(Diethylamino)-5-[(2-octyldecyl)imino]benzo[a]phenoxazine). Chromoionophore II exhibits light absorbance peaks at 520 nm and 660 nm and a fluorescent emission peak at 660 nm. Chromoionophore Ill has light absorbance peaks at 500 nm and 650 nm and fluorescent emission peaks at 570 nm and 670 nm. Essentially any biocompatible nanosensor that is shown to detect the concentration of a specific metabolite, catabolite, protein, drug or other compound may be used.

### Device Calibration and Validation

The primary means of calibrating and validating device operation also serves as an excellent example of the power of the combined experimental and mathematical approach. For example, Matrix spheres (e.g. alginate spheres) encapsulating known concentrations of metabolic enzymes and nanosensors (no cells) can be perfused in the device, generating chemical gradients due to enzymatic action. For example, a bed of microspheres containing glucose oxidase (at constant flow rate and initial oxygen/glucose concentrations) will generate stable gradients in both glucose and oxygen, as both are consumed in the reaction. Measurements of these gradients are fit to the transport model in order to extract oxygen and glucose consumption rates, which can be calibrated against the known enzyme activity and concentration. Analyzing concentration gradients established under different operating conditions (e.g. varying flow rate, initial concentrations, or other parameters as listed herein) will provide precise calibration of both the mathematical model and the sensors, as well as provide important characterization of the dynamics of changing conditions within the device. Further calibration for the ability to measure gradients in metabolic activity (i.e. non-uniform cell concentration or consumption/production rates) by creating devices filled with layers of microspheres with different concentrations of enzyme.

Besides glucose-specific enzymes, a number of other enzymes can employed for monitoring other metabolites in the device. Examples include cholesterol using the enzyme cholesterol oxidase, and lactate, using the enzyme lactate oxidase or lactate dehydrogenase. Such enzyme-metabolite pairs form the basis of different metabolite sensors with each sensor configured to maximize the signal of the target metabolite.

A user may also directly calibrate the integrated system by fitting gradients measured in devices filled with nanoCEMS to extract cellular consumption/production rates, then compare these to cellular metabolic rates determined on isolated cells known methods. As discussed above, measurements of biochemicals in the input and output flow streams provide calibration of in situ nano-sensor and direct optical measurements, while input/output differences provide independent assay of bulk consumption/production rates for calibration of metabolic parameters. Finally, the ability to recover nanoCEMS from defined locations can be tested by filling the device with “bands” of fluorescently-labeled alginate microspheres of different intensities, measuring the fluorescent signal across the device, then harvesting the microspheres and measuring the mean intensity in each recovered sample.

In some embodiments, it may be advantageous to further replicate the extracellular matrix within a nanoCEMS. As shown in FIG. 4, after several days of culture at low density, the cells form 3D colonies within the alginate spheres, thereby establishing more normal cell-cell and cell-ECM contacts. Therefore, more tissue-like matrices, including alginate modified with ECM proteins and ECM-like matrix materials (e.g. Matrigel) can be used to improve cell growth and viability. In another embodiment, alginate spheres are created that contain a single small multicellular spheroid: the alginate will prevent the spheroids from aggregating and allow perfusion throughout the cylinder, while the small spheroid size will minimize internal gradients. These can be created using the droplet device as described herein by externally culturing alginate spheres containing a few cells until they form a single aggregate, then filling the device with these CEMS. In another embodiments, CEMS are cultured at a relatively low cell density to maintain cultures with discrete alginate spheres for as long as it takes for the cells at the top of the cylinder to divide 4-5 times.

### An Optical Detection Platform for Imaging of pH in Perfusion Device.

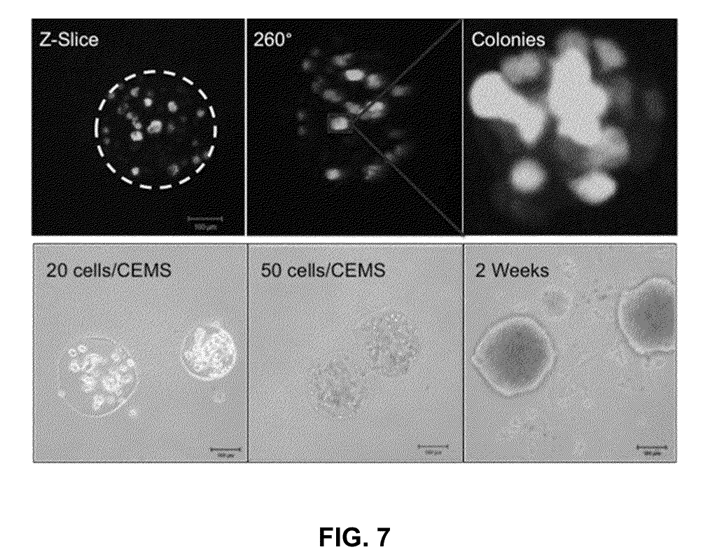

As described below, strategies exist for integrating pH sensitive fluorescent reporters directly into the alginate matrix, or for developing pH sensitive nanoparticles that can, in turn, be integrated into the alginate matrix (see FIG. 7). Thus, a user can compare these two modes of detection in order to understand the relative strengths of each. In some situations, another, non-pH-sensitive fluorophore is integrated into the matrix in order to provide a reference measurement, as is standard practice for luminescence-based detection. Luminescence detection may comprise an epi-illuminated design in which a low-power (e.g., 10×) long working distance objective is illuminated with light either from a filtered white-light source, LED or laser, and detected emission light is separated by filters onto PMT detectors. Imaging can be performed simply by translating the perfusion device along its flow axis under the imaging objective.

In some embodiments, pH detection, can be through a fluorophores such as Fluorescein isothiocyanate (FITC), a widely used pH sensitive dye. An internal reference, can be for example, tetramethylrhodmaine isothiocyanate (TRITC) a pH insensitive dye that emits in a different wavelength range with minimal spectral overlap (Burns et al., Chemical Society Reviews 35: 1028-1042 (2006)). Both of these dyes can be cross-linked into alginate microspheres (or another polymer, biopolymer or combinations thereof as described) with or without embedded cells to form labeled alginate, which can then be integrated with a much higher fraction of unlabeled alginated prior to Ca2+-induced cross linking (Kong et al., Journal of the American Chemical Society 129: 4518-4519 (2007)). This approach will result in formation of cross-linked alginate microparticles, but with responsive fluorophores added for pH interrogation. In further embodiments, a user can use silica encapsulated pH sensitive dyes, such as, but not limited to, Cornell dots as disclosed in Choi et al., (Journal of Biomedical Optics 12 (6), 064007-(1-11) (2007)). Briefly, a core-shell structure can be made in which the TRITC is incorporated into the core of a silica nanoparticle, while the FITC is positioned in the surface layer of the particle. These silica particles are then used for pH measurements, using the ratiometric methods described above. However, silica encapsulation leads to an increase in stability of the fluorophores, greatly reducing any limitations arising from photobleaching. The labeled silica nanoparticles can also be integrated with the polymer matrix, such as alginate, prior to Ca2+-induced cross linking as a means to interrogate local pH in real time throughout the perfusion chamber.

### Near Simultaneous Detection of pH and Dissolved O2 and CO2 Concentrations.

In addition to pH sensing, optical measurement of dissolved 02 concentration may be desirable. Here, a user relies on the intensity change associated with luminescence quenching of transition-metal based chromophores, for example Ru-based bipyridine compounds or Pt-based porphyrin compounds, by dissolved O2 (Schaferling et al., Angewandte Chemie-International Edition 51: 3532-3554 (2012); Wang et al., Journal of the American Chemical Society 134: 17011-17014 (2012)). Likewise, several different groups have worked out methods for fluorescence-based (or fluorescence-lifetime-based) optical detection of dissolved CO2 concentrations (Cajlakovic et al., Analytica Chimica Acta 573: 57-64 (2006); Schroeder et al., Microchimica Acta 158: 205-218 (2007); Wencel et al., Analytical and Bioanalytical Chemistry 398: 1899-1907 (2010)). These approaches are generally more complex, relying upon equilibration of dissolved CO2 through a gas-permeable membrane with a carbonate buffer and pH-sensitive fluorophore, but have been implemented in particle-based assays in a variety of complex biological settings including both bacterial fermenters and mammalian cell culture chambers.

In the case of dissolved O2 concentration, a user can either directly integrate the luminescent transition-metal compound directly into the cross-linked matrix, or incorporating the compound into a silica nanoparticle and integrating the particle into the matrix. The former can be readily achieved, for example in the case of [Ru-(bipyridine)₃]²⁺-based species in that this molecule is available commercially with carboxylated bipyridine ligands, which could simply be incorporated into a matrix (such as alginate) through Ca2+-induced cross linking. Other Ru-based luminescent species can be integrated into silica and porous silica nanoparticles (or thin films), which can then be used as oxygen sensing materials.

In some instances, such as when steady-state conditions are reached in the perfusion device, the time scale on which significant evolution of the chemical microenvironment is likely to occur is at least minutes. Thus, a user can perform effective multiplexed measurement simply by cycling between the different excitation and analysis conditions associated with each detection modality. For example, an optical analysis system, with added mirrors on “flipper” mounts, so that different excitation sources can be directed into the objective and the collected emission can be directed into different analysis paths. In the case of phase-sensitive detection, it may be advantageous to use pulsed LED sources at approximately MHz repetition rates, and use lock-in analysis of the detected signal.

### Raman Spectroscopy Methods for Detection of Small Metabolites

The luminescence-based approaches described above will provide for a straightforward means of examining basic concentration gradients and cellular metabolic processes in the perfusion device. However, it is also desirable to have spatial and time-dependent measurements of many other small molecules such as glucose, lactate, glutamine, L-amino acids and derivatives, acetate, formate, creatine, citrate, succinate, betaine, taurine, ethanol, fucose, galactose, glucitol, glucose, methanol, propylene glycol, acetoacetate, butyrate, glycolate, pthalate, propionate, pyruvate or other metabolites/solutes, or in the case of evaluating treatment efficacy, of potential drug candidates. Accordingly, some embodiments may make use of Raman and surface-enhanced Raman spectroscopy (SERS) as a means of measuring the concentration of these small molecules.

Raman spectroscopy is based on the principle that monochromatic incident radiation on materials will be reflected, absorbed or scattered in a specific manner, which is dependent upon the particular molecule or protein which receives the radiation. While a majority of the energy is scattered at the same wavelength (Rayleigh scatter), a small amount (e.g., 10″7) of radiation is scattered at some different wavelength (Stokes and Antistokes scatter). This scatter is associated with rotational, vibrational and electronic level transitions.

The change in wavelength of the scattered photon provides chemical and structural information.

In certain embodiments, Raman spectroscopy can be performed on multi-component mixtures such as is described herein to provide a highly specific “fingerprint” of the components. The spectral fingerprint resulting from a Raman spectroscopy analysis of a mixture will be the superposition of each individual component. The relative intensities of the signal correlate with the relative concentrations of the particular components. Accordingly, in certain embodiments, Raman spectroscopy can be used to qualitatively and quantitatively characterize a mixture of components.

Raman spectroscopy can be used to characterize most samples, including solids, liquids, slurries, gels, films, powders and some gases, with a very short signal acquisition time. Generally, samples can be taken directly from the bioprocess at issue, without the need for special preparation techniques. Also, incident and scattered light can be transmitted over long distances allowing remote monitoring. Furthermore, since water provides only a weak Raman scatter, aqueous samples can be characterized without significant interference from the water.

The applicable processes and compositions described herein can be analyzed based on commercially available Raman spectroscopy analyzers. For example, a RamanRX2™ analyzer, or other analyzers commercially available from Kaiser Optical Systems, Inc. (Ann Arbor, Mich.) can be employed. Alternatively, Raman analyzers commercially available from, for example, PerkinElmer (Waltham, Mass.), Renishaw (Gloucestershire, UK) and Princeton Instruments (Trenton, N.J.). Technical details and operating parameters for the commercially available Raman spectroscopy analyzers can be obtained from the respective vendors.

In some embodiments, the measurement of glucose concentrations in biological settings using Raman spectroscopy is desired. Accordingly, Raman spectroscopy can provide a reliable measurement of glucose concentrations at physiological ranges. Some embodiment may use a 785 nm diode laser as the excitation source, and measuring the inelastically scattered light on a dispersive spectrometer equipped with a liquid-nitrogen cooled CCD detector.

One limitation of standard Raman scattering is that the signal rates are significantly lower than for the fluorescence and luminescence methods applied above. It is likely that several minutes of data acquisition will be required to obtain spectra of sufficient signal-to-noise to allow glucose concentration determination, where the concentration determination may require using a principal-component analysis approach based on data sets obtained under known glucose levels, which can be measured independently at the inlet and outlet (In/Out Assay). The degree to which this limitation affects the ability to monitor changes in the cell culture device depends on the time scale on which concentration changes might occur. Therefore, some embodiments can employ the use of surface-enhanced Raman spectroscopy (SERS) to detect a molecule of interest as described herein, such glucose concentrations. In SERS, an enhancement of several orders of magnitude in Raman scattering strength is obtained near the surface of metal nanoparticle or near the junctions of assemblies of metal nanoparticles. The metal or other enhancing surface will couple electromagnetically to incident electromagnetic radiation and create a locally amplified electromagnetic field that leads to 102-to 109-fold or greater increases in the Raman scattering of a SERS active molecule situated on or near the enhancing surface. The output in a SERS experiment is the fingerprint-like Raman spectrum of the SERS active molecule.

SERS and similar techniques can be implemented with particles such as nanoparticles as described herein. In some embodiments, gold or silver nanoparticles comprise a SERS enhancing surface, and gold colloid may be suspended in a mixture to provide for enhanced Raman spectrum detection. SERS may also be performed with more complex SERS-active nanoparticles, for example SERS nanotags, as described in U.S. Pat. No. 6,514,767, U.S. Pat. No. 6,861,263, U.S. Pat. No. 7,443,489 and elsewhere.

In a specific embodiment, silver or other metallic nanoparticles are directly crosslinked to the polymer matrix, and SERS signals are measured to determine glucose concentrations such as disclosed in Shah et al., (Anal. Chem., 79, 6927-6932 (2007)). This method allows the ability to detect glucose on a time scale that will closely match the time scale of the luminescence measurements.

### Optical Methods for the Cellular Microenvironment

In addition to the measurement of chemical microenvironments using luminescent, nanoparticle or spectroscopic tools, the direct measurement of cellular properties will also provide important information. Essentially, any cellular assay or labeling strategy that can be implemented in other cell culture platforms can also be introduced into the perfusion device. Some embodiments can employ measurements of polarized elastic light scatter to optically measure cell concentration. This technique can be used to detect cells and even differentiate between tumor/normal cells and cells that are proliferating versus arrested cells (Mourant et al., Journal of biomedical optics 7: 378-387 (2002); Ramachandran et al., Optics express 15: 4039-4053 (2007)). Optical measurements of cells can be calibrated by comparison to assays on cells obtained from dissociated nanoCEMS following extrusion. Moreover, the presence of nanoparticles in the nanoCEMS will provide a discrete backscatter signal that can be used to calibrate the cellular signals.

A complimentary approach to optical cell assay is to use cells expressing fluorescent proteins, in which case direct fluorescence measurements can be used to determine cell concentrations. Even in cells that “constitutively express” fluorescence proteins, the expression level has been shown to change under microenvironmental stress. An example of a fluorescent protein includes green-fluorescent-protein (GFP) tagged proteins and its derivatives.

A third optical detection method involves pre-labelling cells with a fluorescent tracking dye that incorporates irreversibly in the cell membrane. Such dyes are stable over long periods (weeks) and have the added advantage of measuring cellular proliferation by the reduction in signal per cell following division (Chadli et al., Methods Mol Biol 989: 83-97 (2013); Makinen et al., J Neurosci Methods 215: 88-96 (2013)). In other embodiments, cells can be labeled with any fluorophore or material that produces a detectable signal. Preferably, if multiple labels are used, the labels are configured to be detected, and measure a microenvironmental parameter independently of one another (e.g. labels that fluoresce at different wavelengths).

Regardless of the technique used, the in situ optical signals fluorescent protein and membrane labels can be calibrated by comparison to flow cytometry of cells harvested from different regions.

### Transport/Metabolism/Physiology Models

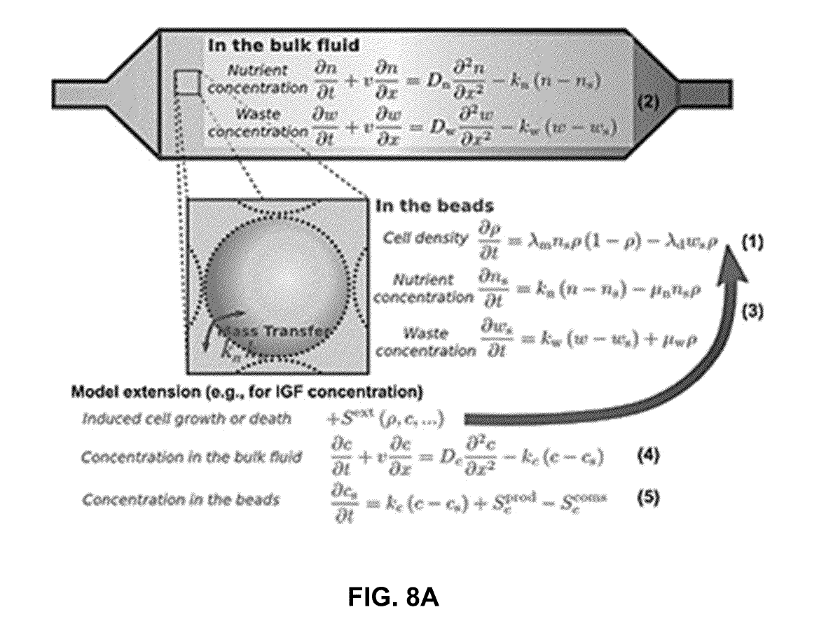

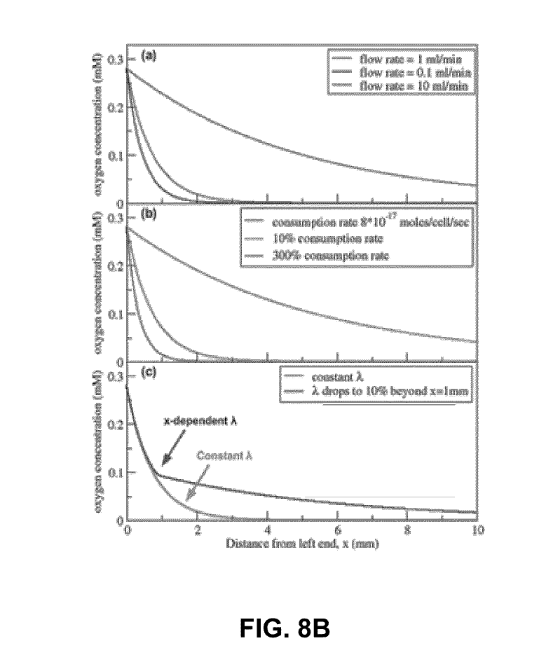

Applicant disclose herein idealized methods from physics and engineering to describe the transport processes occurring within the perfusion device. Here, Applicant discloses a model the system based on continuum approach to capture the dynamics of the cells, nutrients (such as amino acids, thiamine, hypoxanthine, folic acid, biotin, pantothenate, choline, inositol, niacinamide, pyroxidine, riboflavin, thymidine, cyanocobalamin, pyruvate, lipoic acid, glucose and various metals and inorganic ions) and waste products (such as lactic acid, carbon dioxide, peptides, enzymatic breakdown products of proteins and lipids, sugars etc.) as well as the interfacial mass transport between the spheres and medium (see FIGS. 8A, 8B). It is initially assumed that the adiabatic packed bed is composed of spherical beads (100 μm in diameter) that are uniformly distributed throughout the reactor along the major axis. It is also assumed that the time scale of intra-bead diffusion is much shorter than that of the diffusion and convection occurred within the device. Thus, the cell culture model is based on a logistic growth models:

$$\frac{\partial ρ}{\partial t} = {λ}_{m} {n}_{s} ρ ( 1 - ρ ) - {λ}_{w} {w}_{s} ρ ,$$

for cell volume fraction p, and advection/diffusion/reaction equations to describe the nutrient and waste transport

$$\frac{\partial n}{\partial t} + v \frac{\partial n}{\partial x} = {D}_{n} \frac{{\partial }^{2} n}{\partial {x}^{2}} - {k}_{n} ( n - {n}_{s} ) , \\ \frac{\partial w}{\partial t} + v \frac{\partial w}{\partial x} = {D}_{w} \frac{{\partial }^{2} w}{\partial {x}^{2}} - {k}_{w} ( w - {w}_{s} ) ,$$

for nutrient concentration n and waste concentration w in the bulk medium fluid.

The following reaction equations,

$$\frac{\partial {n}_{s}}{\partial t} = {k}_{n} ( n - {n}_{s} ) - {μ}_{n} {n}_{s} ρ , \\ \frac{\partial {w}_{s}}{\partial t} = {k}_{n} ( w - {w}_{s} ) + {μ}_{w} ρ ,$$

will be used to describe the consumption of the nutrient concentration (nₛ) and the production of waste (wₙ) inside the matrix spheres. The basic set of model parameters consist of cell mitosis rate (λₘ) in response to nutrient and growth factors, cell death rate (λ_{d}) due to surrounding waste and/or drug, the nutrient consumption rate by cells (μₙ), and waste production rate by cells (μ_{w}), as well as the mechanical parameters, such as the advection speed (v), the diffusivities of nutrient (Dₙ) and waste (D_{w}), and the mass transfer rates of nutrient (kₙ) and waste (kw) between the bulk medium fluid and the matrix spheres. Note that the form of the diffusion/waste equations is consistent with the time-dependent heterogeneous packed-bed reactor model with axial dispersion. Other functions, e.g., Michaelis-Menten kinetics, can also be used to describe the consumption of nutrients as indicated by experimental studies.

Assuming that the cells evolve at a time scale much slower than the nutrient/waste distributions, it is a fair approximation that, for an initial short period of time, the cell distribution is homogeneous, all the rate parameters are constant, and the nutrient/waste distributions quickly settle to a steady state. Using idealized boundary conditions (at x=0, n(x)=1, w(x)=0, dw/dx=0; at x=+∞, dn/dx=0), the steady-state solution can be analytically obtained for the nutrient distribution n(x)˜exp([v2+4Dnμ′n)½]×/(2Dn)) and the waste distribution w(x)˜μwx/v, where μ′n=μn/(1−μn/kn) is the adjusted nutrient consumption rate taking the mass transfer process into consideration. As previously stated, the advection speed, v, is a controllable parameter of the perfusion device. The optical nanosensor data collected for this initial short period can be used to validate the dependence on v in the steady-state solution.

At a later time (presumably after 24 hrs, i.e., a typical cell-cycle duration; this time-length differs in different cell types), the cell volume fraction, ρ, becomes heterogeneous due to cell proliferation and death. Furthermore, it is known that cell metabolic rate varies in response to changes in the microenvironment. Hence, the nutrient consumption rate, pn, and the waste production rate, pw, can have spatial dependence due to the spatial variation of nutrients and wastes. Optical nanosensor data can be used as inputs for numerical simulation of the cell culture model to back calculate pn and pw as spatial functions. As a result, Applicant can estimate how the metabolic rates depend on the spatial variations of microenvironmental variables. In some situations, and to more accurately describe the dynamics of the perfusion device in the numerical simulation, a user may adopt an inhomogeneous boundary condition or a Danckwerts boundary condition depending on how the flow is controlled in the experiment.

The basic set of the model can be further expanded to corroborate the investigation of more sophisticated cell culture dynamics that may be conducted using the microenvironmental gradient device. One example is a drug delivery model to investigate the diffusion barrier of drug efficacy using the device. A pharmacokinetics/pharmacodynamics model can be adapted to incorporate packed bed reactor (PBR) characteristics and add to the calibrated basic model to assist such investigations. Another example is a competition model of two types of cell species in a changing microenvironment. The differences in cell proliferation/death rates as well as nutrient uptake rate and waste production rate can readily be incorporated into the mathematical model. Interactions between the cell species can either be added directly as additional mass exchange terms, or be described indirectly by additional advection/diffusion/reaction equations of signaling molecules, depending on the experimental data obtained.

### Definitions

One skilled in the art may refer to general reference texts for detailed descriptions of known techniques discussed herein or equivalent techniques. These texts include Current Protocols in Molecular Biology (Ausubel et. al, eds. John Wiley & Sons, N.Y. and supplements thereto), Current Protocols in Immunology (Coligan et al, eds., John Wiley St Sons, N.Y. and supplements thereto), Current Protocols in Pharmacology (Enna et al, eds. John Wiley & Sons, N.Y. and supplements thereto) and Remington: The Science and Practice of Pharmacy (Lippincott Williams & Wilicins, 2Vt edition (2005)), for example.

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations. The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the end-points of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The following Examples are intended to illustrate the above invention and should not be construed as to narrow its scope. One skilled in the art will readily recognize that the Examples suggest many other ways in which the invention could be practiced. It should be understood that numerous variations and modifications might be made while remaining within the scope of the invention.

### EXAMPLE 1

### Formation and Use of a 3-D Tissue Model Using Multiple Cell Types

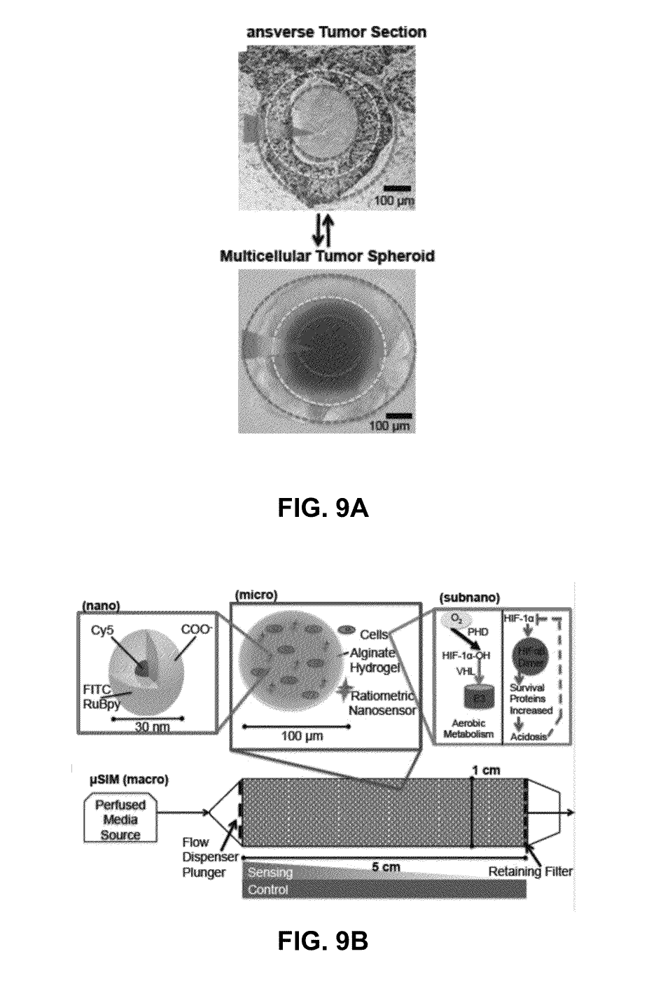

Adaptation of cancer cells to an evolving microenvironment is a crucial aspect of malignant progression. Cells depend on vascular nutrient and waste transport to maintain metabolic and physiological requirements. However, increasing tumor mass creates multifactorial gradients in extracellular pH (pHe), extracellular oxygen (O2), and the transcription factor hypoxia inducible factor la (HIF-1a). Exactly how these gradients are regulated and their effects on cellular proliferation, metabolism and viability are currently open questions. Producing uniform and controllable in vitro 3D perfusion models of tumors with integrated chemical nanosensing capabilities is critical for discovering how interacting microenvironmental parameters (pHe and O2) influence cellular pathophysiology (HIF-1a). Improved methods are needed for generating such 3D models containing multiple cell types and multiplex chemical nanosensors for controllable in situ assays of chemical, metabolic and physiological microenvironments. We are developing new instrumentation that will provide spatially correlated measurements of these gradients, allowing mechanistic investigation of the relationships between pHe, O2, and HIF-la (FIGS. 9A, 9B).

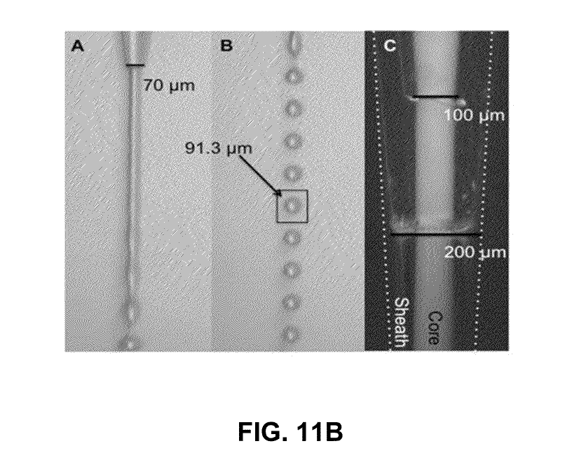

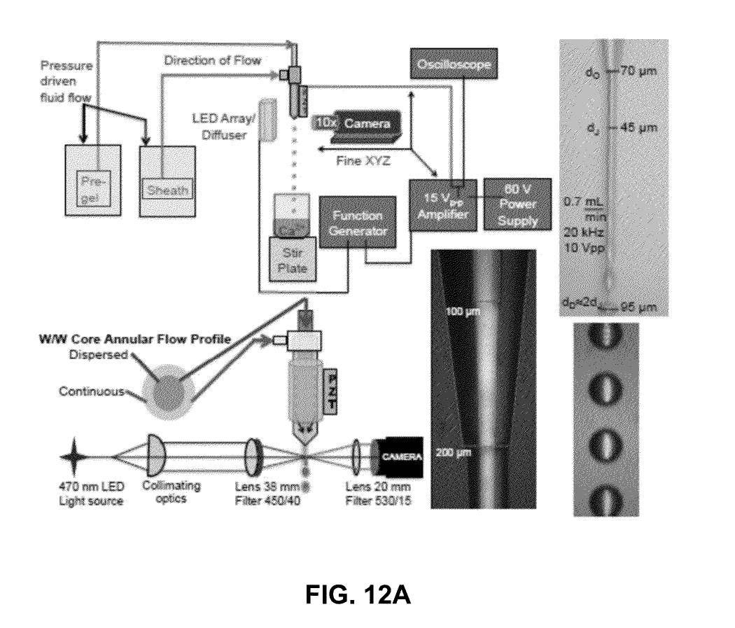

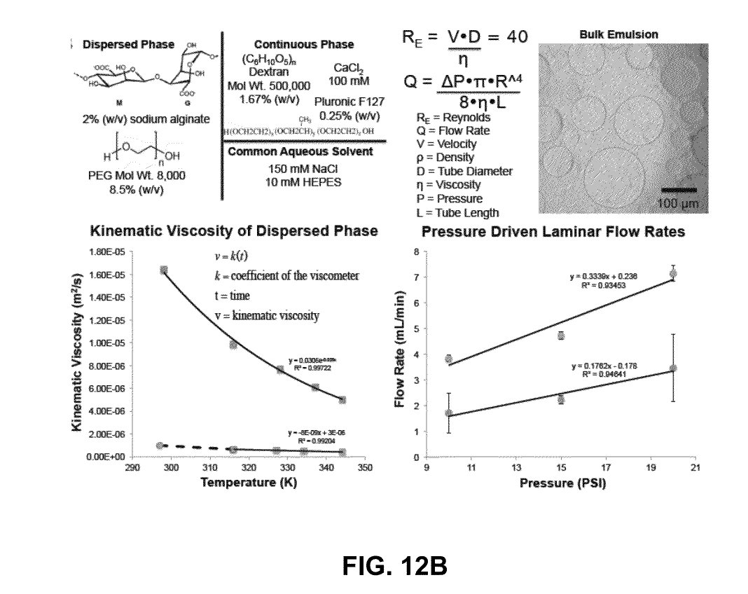

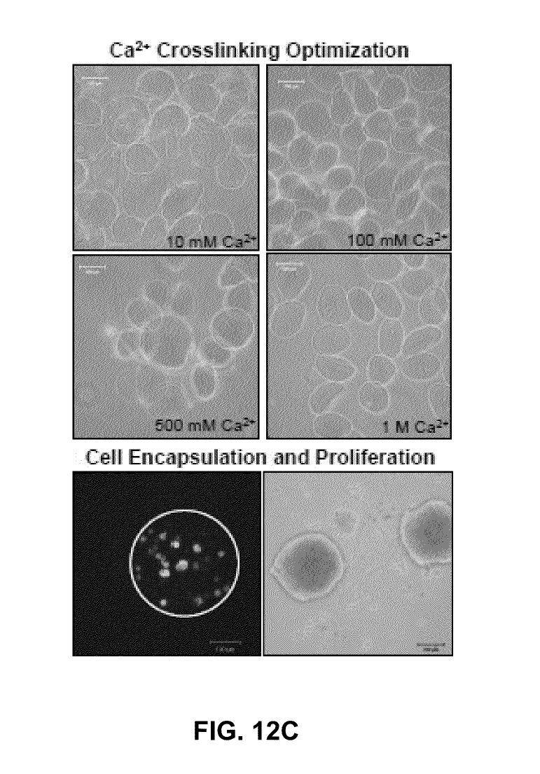

Referencing FIGS. 11-12, we have developed a high-throughput instrument that builds upon the concepts of cell sorting to produce uniform, cell-encapsulating microspheres with integrated optical nanosensors. The instrument uses microfluidic devices with defined orifice diameters (70-200 μm), pressure driven fluid flow of viscous solutions, and high frequency acoustics tuned to produce uniform droplets in air. The cell-encapsulating biomaterial consists of sodium alginate (ALG) and polyethylene glycol (PEG) in buffer. The ALG/PEG droplets crosslink by introduction of calcium ions in a solution that contains interface stabilizing chemicals, dextran (DEX) and Pluronic F-127. Varying the orifice diameter, the flow velocity (up to 10 mL/min) and piezoelectric frequency (1-40 kHz) controls the size of the droplets in air (70-300 μm diameters). In other embodiments, the devices use two nesting glass capillaries to initiate crosslinking of droplets in air. The device generates droplets between 2-20 kHz, depending on flow rates. In roughly seven minutes enough droplets (5×106) are produced to fill a 1 cm×1 cm×5 cm perfusion device.

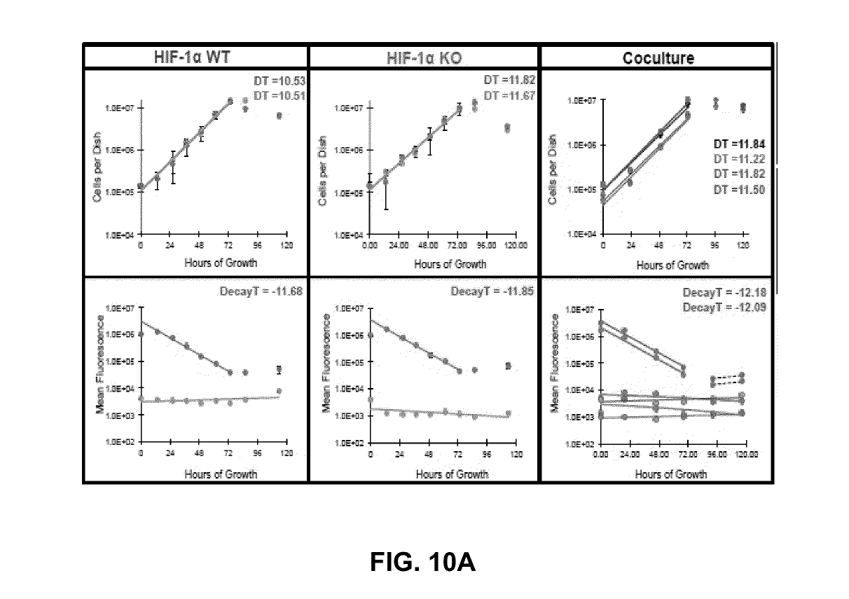

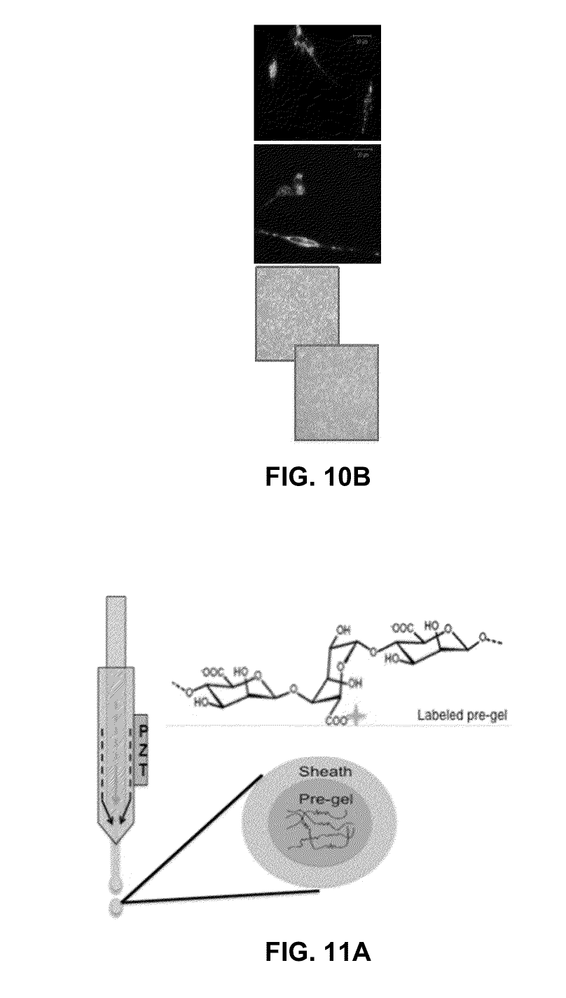

In order to determine initial cell-encapsulation concentrations for droplets initiated without internal microenvironmental gradients, we measured the doubling times of HIF-1a wild type and knockout cells in monolayer co-culture using green (WT) or red (KO) membrane dyes. Using flow cytometry to identify the two cell types in a mixture provides the ability to measure growth in co-culture by both dye dilution and differential counting. In uniform culture conditions, the cells have doubling times of 11-13 hours when measured by cell counts or dye dilution. Moreover, use of this WT/KO co-culture exposes the two cell types to identical microenvironmental parameters, allowing us to identify the combinatorial range of [O₂] and pHe in which HIF-1α is active or inhibited (FIGS. 10A, 10B).

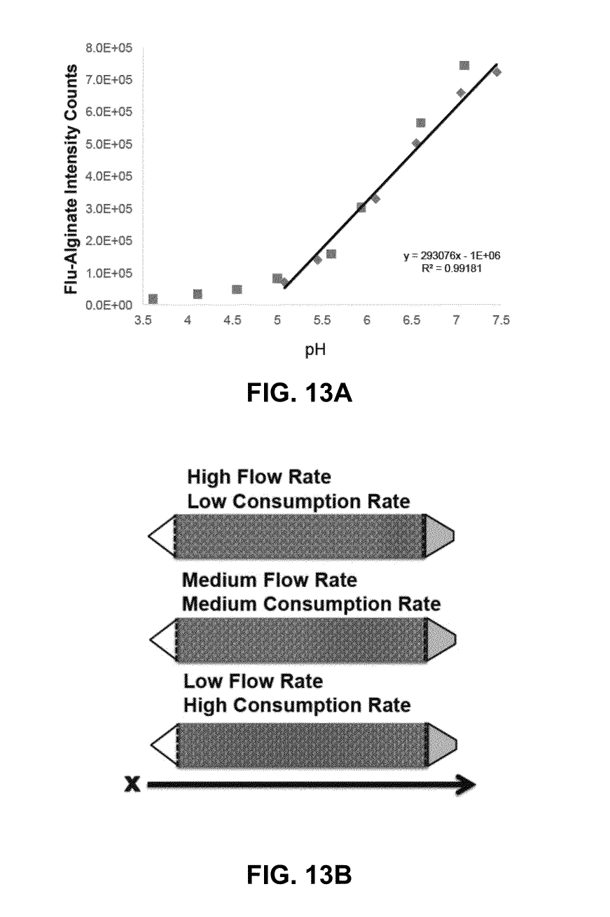

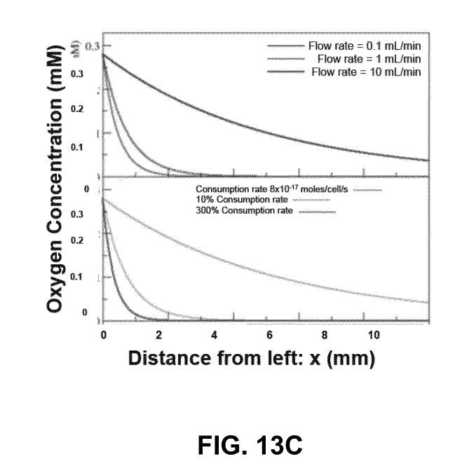

Such a system of fabricating perfusion devices with uniform nanoCEMS allows, for example, investigating the coupled and uncoupled effects of hypoxia and acidosis by setting initial conditions to produce gradients such that cells are exposed to hypoxia without acidosis, acidosis without hypoxia and combined gradients. We have control over the total perfusion flow rate, medium buffering capacity to control the pHe gradient, and increasing medium O₂ to achieve known threshold concentrations at the end of the column (FIGS. 13A, 13B, 13C).

These results are suggest the design and implementation of a droplet-packed perfusion cell culture system will produce a controllable and quantitative model of the chemical, metabolic and physiological gradients produced by cellular metabolism in a tumor.

### EXAMPLE 2

### Measurement of HIF-1α Activity and Survival in Combinatorial pHₑ and O₂ Environments.

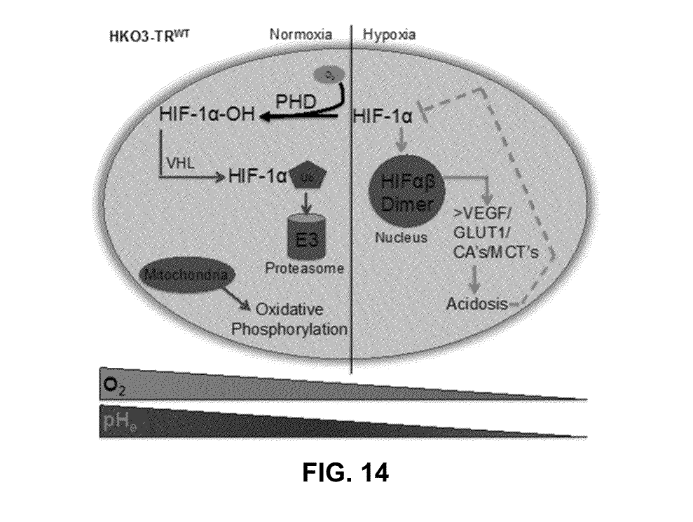

The control of tumor cell proliferation, survival, and metabolism in acidic and hypoxic microenvironmental conditions has been a controversial topic in cancer biology. Direct measurements of intra-tumoral pHₑ and O₂ levels have shown extremely heterogeneous distributions of both parameters at the micro-regional level. However, due to technical limitations, comprehensive in vitro studies have not been feasible. A rapidly proliferating tumor in vivo has large variations in nutrients and wastes due to spatially expanding margins, insufficient transport due to chaotic vascularization, and dysregulated metabolism. Cancer cells survive stressed microenvironments by selecting phenotypes that enhance survival including suppressing apoptosis, initiating angiogenesis, and enabling a switch in energy metabolism. A key player in cellular sensing of changing extracellular conditions is the transcription factor HIF-1α (hypoxia-inducible factor-1α). The central role of this transcription factor in cellular response to hypoxia is illustrated in FIG. 14. However, it is unclear how the micro regional distribution of HIF-1α is modulated by coupled or uncoupled gradients in hypoxia and acidosis. This transcriptional response element controls the hypoxia induced, adaptive metabolic switch from oxidative phosphorylation to glycolysis, which results in the accumulation of lactic acid. It was once thought that acidosis was only toxic to cells, but it is now clinically recognized that mild acidosis (pH 6.5 and above) is protective. Lactic acid can indirectly stabilize HIF-1α and is thought to perpetuate the activation of HIF-1 independent of hypoxia, but due to technical limitations the mechanism has yet to be determined. An increase in the activity of HIF-1α and subsequent metabolic programs alters the generation of pHₑ and O₂ gradients by cancer cells and directly influences survival. Therefore, more aggressive cancer cell phenotypes evolve in order to survive stressed microenvironments.

Chemoresistance is not characteristic of all hypoxia-exposed cells. HIF-la activity is implicated in therapy resistance due to pro-tumor effects, although some changes observed in hypoxic cells can result in increased drug sensitivity. The development of interventions that aim to target cancer by pHₑ disruption requires an improved understanding of how altered microenvironmental conditions influence adaptive phenotypes. This new ability to measure cellular HIF-1α activity as spatially correlated to environmental pHₑ and O₂ will improve our understanding of the tumor microenvironment, therapy resistance, and drug sensitivity. The existing lack of information results from the failure of current experimental systems and models to have complete control and analysis capabilities over multiple microenvironmental gradients. Here, using an embodiments of the present disclosure, Applicant demonstrates there are combined low pHₑ and low O₂ conditions that inhibit HIF-1α mediated metabolic adaptations, thereby reducing cell survival. These experiments give valuable understanding of the effects of acidic, hypoxic, and nutrient deprived environments on tumor cell proliferation, metabolism and survival.

The advantage of investigating our hypothesis in the μSIM instrument, as opposed to using conventional cell culture techniques, is the ability to spatially resolve the dynamic evolution of cellular heterogeneity in 3D with control over interacting parameters. To address the hypothesis, Applicant used c-myc transformed mouse embryonic fibroblast HIF-1α wild type cells (HKO3-TR^{WT}) and also HIF-1α null phenotype (HKO/3-TR^{NULL}) cells. Applicant encapsulated 2.5×10⁷ cells total, corresponding to 5 cells each in 5×10⁶ droplets, to achieve the sphere packing in the pSIM perfusion column. Applicant measured the doubling time of each of these cell lines to be ˜12 hours in monolayer cultures. Applicant anticipates that ˜7 doublings will yield enough cells for the downstream analysis but not generate internal droplet gradients. Applicant investigated the coupled and uncoupled effects of acidosis and hypoxia for each indicated cell type as 3D monocultures by initiating the pSIM instrument with eight total combinatorial conditions: pHₑ threshold at 7.4/ΔO₂, O₂ threshold at 60 mmHg/ΔpHₑ, pHₑ/O₂ maintained at respective thresholds, and ΔpHₑ/ΔO₂. The combinatorial microenvironments are generated by manipulating the buffering capacity of the media for pHₑ and by altering the input O₂ concentration. To uncouple the pHₑ and O₂ gradients, the conditions are independently held above a threshold value in which HIF-1α is known to not be transcriptionally active. The use of HIF-1α null cell lines in the μSIM instrument will allow us to conclude that observed differences in proliferation, survival, and metabolism (as a function of the combinatorial microenvironments) is due to HIF-1α modulation. Additionally, the pSIM instrument will be initiated with a co-culture of the HK03-TR^{WT} and HKO/3-TR^{NULL} cells with membranes stained red and green, respectively. The μSIM co-culture approach will directly expose the two cell types to identical microenvironmental parameters. The HKO/3-TR^{NULL} cells in co-culture are an internal control that will comparatively identify the combinatorial range of pHₑ and O₂ in which HIF-1α transitions from transcriptionally inactive to active.

For the experiment, mono- or co-cultures will be established in the device and the initial conditions (medium composition and flow rates) will be manipulated to establish the eight culture conditions described above. We will measure the extracellular pHₑ and O₂ gradients in situ in μSIM as well as quantifying overall metabolic flux of the cultures. After a defined period, the biochemical microenvironments and cells will be fractionated from the device and processed to measure cytoplasmic HIF-1α (fluorescence antibody detection using Western Blot analysis and flow cytometry), proliferation (membrane dye dilution assays and DNA content analysis), survival (flow cytometry for VEGF, Live/Dead assay, and apoptosis assay), and metabolism (flow cytometry custom bead array for GLUT1, CA's, MCT's). It is expected that a significant difference in HIF-la, proliferation, survival, and metabolism (using the metrics indicated above) for the device that contains the HK03-TR^{WT} cells in monoculture, and acidic/hypoxic conditions (ΔpHₑ/ΔO₂) as compared to the devices that were held at combinatorial thresholds in the pHₑ/O₂ environments. In the co-culture experiment, we expect to measure the differential values for pHₑ and O₂ that facilitate the activation of HIF-1α. Ultimately, Applicant identifies the spatially discrete ranges of acidosis and hypoxia that reduce the half-life of cytoplasmic HIF-1α mitigating any pro-tumor effects.

This is the first time a 3D in vitro cell culture contains controlled and quantified gradients of pHₑ/O₂, and correlates the measurements to heterogeneous cellular phenotypes analyzed over space and time. The ability to perform such comprehensive studies will have widespread effects on cancer etiology, therapeutic strategies, and the understanding of chemoresistance. An effective treatment targeting cancer cell pHₑ/O₂ regulation could exploit the possible downregulation of HIF-1α activity in acidic conditions, thereby decreasing induction of pro-tumor proteins that mediate survival in the established hypoxic microenvironment.

While the disclosure is susceptible to various modifications and alternative forms, specific exemplary embodiments of the present invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

What is claimed is:

1. A perfusion device comprising:
a chamber body having an input and an output;
a removable screen adjacent the output;
a plunger and screen; and
a first layer of inert microspheres and a second layer of inert microsphere, and a layer of nanosensor-cell embedded matrix spheres (nanoCEMS) disposed there between, wherein the nanoCEMS comprise a crosslinked polymer matrix with entrapped cells and nanosensors.

2. The perfusion device of claim 1, wherein addition of a molecule to the input establishes a gradient of the molecule throughout the perfusion device.

3. The perfusion device of claim 1, wherein the crosslinked polymer matrix comprises alginate.

4. The perfusion device of claim 1, wherein the entrapped cells comprise normal cells, stem cells, immortalized cells, cancer cells, genetically engineered cells, patient derived cells or a combination thereof.

5. The perfusion device of claim 1, wherein the entrapped cells comprise two or more different cell types in a co-culture, wherein the different cell types have a detectable label configured so the microenvironmental effects on the different cell type are determined independently of one another.

6. The perfusion device of claim 1, wherein the nanosensor is a fluorophore, nanoparticle, electrode, quantum dot or Cornell dots.

7. The perfusion device of claim 1, wherein the nanosensor detects oxygen concentration, carbon dioxide concentration, pH levels, metabolites, catabolites, secreted proteins, or ligand binding.

8. The perfusion device of claim 7, wherein the metabolite selected from the group consisting of glucose, lactate, or glutamine.

9. A method for measuring chemical and cell microenvironments comprising:
perfusing a fluid through the device of claim 1; and
adding a biological compound to the fluid to modify the chemical and cell microenvironments of the nanoCEMS by exposing the nanoCEMS to the biological compound.

10. The method of claim 9, comprising the additional step of measuring the concentration of one or more solutes secreted by the nanoCEMS in response to exposure to the biological compound.

11. The method of claim 9, wherein the cells comprise normal cells, stem cells, immortalized cells, cancer cells, genetically engineered cells, patient derived cells or a combination thereof.

12. The method of claim 9, wherein the nanosensor is a fluorophore, nanoparticle, quantum dot, electrode or Cornell dot.

13. The method of claim 9, wherein the nanosensor detects oxygen concentration, carbon dioxide concentration, pH levels, metabolites, catabolites, nutrients, waste products, secreted proteins, or ligand binding.

14. The method of claim 12, wherein the nanosensor produces a signal detectable by direct optical interrogation.

15. The method of claim 9 further comprising the step of extruding the nanoCEMS and subjecting the extruded nanoCEMS to a biological assay.

16. The method of claim 10, wherein the concentration of one or more solutes is a metabolite concentration or a waste product concentration.

17. The method of claim 16, wherein the metabolite concentration or the waste product concentration is measured in bulk fluid in the device and is determined by the formulas:
$$\frac{\partial n}{\partial t} + v \frac{\partial n}{\partial x} = {D}_{n} \frac{{\partial }^{2} n}{\partial {x}^{2}} - {k}_{n} ( n - {n}_{s} ) , \\ \frac{\partial w}{\partial t} + v \frac{\partial w}{\partial x} = {D}_{w} \frac{{\partial }^{2} w}{\partial {x}^{2}} - {k}_{w} ( w - {w}_{s} ) ,$$
for the metabolite concentration and the waste product concentration, respectively.

18. The method of claim 16, wherein the metabolite concentration or the waste product concentration within the nanoCEMS is determined by the formulas;
$$\frac{\partial {n}_{s}}{\partial t} = {k}_{n} ( n - {n}_{s} ) - {μ}_{n} {n}_{s} ρ , \\ \frac{\partial {w}_{s}}{\partial t} = {k}_{n} ( w - {w}_{s} ) + {μ}_{w} ρ ,$$
for the metabolite concentration and the waste product concentration, respectively.

19. The method of claim 16, wherein a steady-state metabolite concentration or a steady-state waste product concentration is determined by the formula:
n(x)˜exp([v−(v2+4Dnμ′n)½]×/(2Dn)), and
w(x)˜μwx/v
for the steady-state metabolite concentration and the steady-state waste product concentration, respectively.

20. A method for producing matrix spheres comprising;
providing a polymer mixture comprising at least one polymer, and an ionic crosslinking mixture;
pumping the mixtures into a pressurized capillary, the pressurized capillary having an inner chamber with an inner dispensing nozzle and an outer chamber with an outer dispensing nozzle, and the pressurized capillary being coupled to a piezoelectric transducer;
mixing the polymer mixture and the crosslinking mixture upon dispensing from the inner dispensing nozzle and the outer dispensing nozzle;
crosslinking the polymer mixture to form the microspheres;
capturing the microspheres in a receiving solution; and
wherein size of the microspheres is controlled by vibrations of the piezoelectric transducer.

21. The method of claim 20 wherein the polymer is alginate.

22. The method of claim 20, wherein the polymer mixture further comprises a cell, wherein the cell is a normal cells, stem cells, immortalized cells, cancer cells, genetically engineered cells, patient derived cells or a combination thereof.

23. The method of claim 20, wherein the polymer mixture further comprises a nanosensor, wherein the nanosensor is a fluorophore, nanoparticle, quantum dot or Cornell dots.

24. The method of claim 23, wherein the nanosensor is conjugated to the polymer.

25. The method of claim 20, wherein the piezoelectric transducer vibrates to produce droplets in a range of 2-10 kHz.
